(19) United States
(12) Patent Application Publication
(10) Pub. No.: US 2026/0060903 A1
(43) Pub. Date: Mar. 5, 2026

(54) BIODEGRADABLE SPHERICAL PARTICLES FOR COSMETIC APPLICATIONS

(71) Applicant: Momentive Performance Materials Inc., Niskayuna, NY (US)

(72) Inventors: Polina TSUPKO, Bronx, NY (US); Amar PAWAR, Briarcliff Manor, NY (US); Benjamin FALK, Yorktown Heights, NY (US); Sibin WANG, Woodside, NY (US); Rajendar NUTENKI, Telangana (IN)

(21) Appl. No.: 19/314,054

(22) Filed: Aug. 29, 2025

(30) Foreign Application Priority Data
Aug. 30, 2024 (IN) 202411065713

(51) Int. Cl. A61K 8/02; A61K 8/81; A61Q 19/00

(52) U.S. Cl. CPC A61K 8/025; A61K 8/8111; A61Q 19/00; A61K 2800/10; A61K 2800/31

(57) ABSTRACT

The present disclosure provides a composition comprising polymer particles, wherein the polymer particles comprise at least one terpene polymer, as well as methods of preparing the particles. The present disclosure also relates to personal care compositions comprising said particles and methods of using such compositions of polymer particles for treating hair, skin, and nails.

### FIELD

The present disclosure provides a composition comprising polymer particles, wherein the polymer particles comprise at least one terpene polymer, as well as methods of preparing the particles. The present disclosure also relates to personal care compositions comprising said particles and methods of using such compositions of polymer particles for treating hair, skin, and nails.

### BACKGROUND

The personal care industry thrives on being able to deliver personal care formulations that provide multiple performance benefits based on mixtures of several components, with each component providing performance characteristics that impart an important and/or desirable effect to the final formulation. Formulations comprising particles as an important component for delivering key benefits are used in beauty and cosmetics products, especially in products left on the skin, face, and/or hair. The main role of these particles is to provide improved texture to cosmetic formulations, to achieve desirable optical effects such as line-blurring, matte effect, and/or to improve sensory feel during and after use of such cosmetics. Line-blurring, or soft focus effect, is an optical aberration caused by the particles maximizing the diffused light transmission and the scattered reflection, which leads to a minimization of the appearance of imperfections in the skin, face, and/or hair.

Examples of particles currently available in the market and commonly used in personal care formulations comprise polysaccharides (e.g., starch and cellulose based particles), polyesters, acrylates (e.g., poly(methyl methacrylate) microspheres (PMMA)), polysiloxanes (e.g., polymethylsilsesquioxane particles), and polyethylenes. Polysaccharides and polyesters are typically derived from plant-based monomers and oftentimes absorb moisture and oils that change the size and refractive index of the particles. This change leads to undesirable effects in the formulations. Acrylates and polyethylenes are produced from petroleum based monomers, and fall under the category of microplastics, which are expected to be phased out in favor of environmental friendly plant-derived products.

Recently a variety of terpenes and their derivatives have been investigated as natural sources of monomers, to produce various classes of polymers, such as poly(myrcene), poly(limonene), and poly(β-pinene). These terpene polymers are used as tackifier resins in adhesives and are synthesized as pallets or as irregularly shaped powders.

U.S. Pat. No. 3,314,981 discloses the polymerization of turpentine or fractions thereof to provide liquid terpene polymers for use in cosmetics as softening agents. Because the resultant terpene polymers are liquid, they do not possess a spherical shape. U.S. Pat. No. 8,252,270 discloses terpene resins having a molecular weight from about 300 g/mol to about 2,000 g/mol used to prepare a cosmetic composition.

There is a need to provide polymer particles derived from plant-based monomers that provide desirable optical effects such as line-blurring, matte effect, as well as improved sensory aesthetics when used in cosmetic formulations.

### SUMMARY

The present disclosure provides a composition comprising polymer particles, wherein the polymer particles comprise at least one terpene polymer and wherein the polymer particles have a sphericity of at least 0.95 and a Dv50 particle size from about 0.5 μm to about 100 μm.

In some aspects, the at least one terpene polymer of the composition comprising polymer particles is obtained by polymerizing at least one terpene monomer selected from the group consisting of a monoterpene, a sesquiterpene, a diterpene, a sesterterpene, a triterpene, a sesquaterpene, a tetraterpene, and combinations thereof.

In some aspects, the at least one terpene monomer of the composition comprising polymer particles is a monoterpene selected from the group consisting of myrcene, ocimene, alloocimene, L-limonene, D-limonene, α-pinene, and β-pinene.

In some aspects, the at least one terpene polymer of the composition comprising polymer particles is a homopolymer obtained by polymerizing α-pinene, β-pinene, L-limonene, or D-limonene.

In some aspects, the at least one terpene polymer of the composition comprising polymer particles is a copolymer obtained by polymerizing α-pinene, β-pinene, L-limonene, D-limonene, or combinations thereof.

In some aspects, the at least one terpene polymer of the composition comprising polymer particles is hydrogenated from about 0.1% to 100%.

In some aspects, the at least one terpene polymer of the composition comprising polymer particles is a copolymer further comprising at least one monomer that is not a terpene or the hydrogenation product of a terpene.

In some aspects, the at least one terpene polymer has a weight average molecular weight of greater than or equal to about 200 g/mol.

In some aspects, the at least one terpene polymer has a weight average molecular weight from about 200 g/mol to about 20,000 g/mol.

In some aspects, the at least one terpene polymer has a softening point of greater than or equal to about 50° C.

In some aspects, the at least one terpene polymer has a softening point of greater than or equal to about 80° C.

In some aspects, the polymer particles have a Dv50 particle size from about 1 μm to about 50 μm.

In some aspects, the polymer particles have a sphericity of at least 0.98.

In some aspects, the polymer particles are hollow.

In some aspects, the polymer particles are solid.

In some aspects, the polymer particles are porous.

In some aspects, the polymer particles are non-porous.

In some aspects, the polymer particles are smooth.

In some aspects, the polymer particles are insoluble in non-polar oils.

In some aspects, the composition comprising polymer particles is formulated for application to a keratinous substrate, to a fabric, or to leather.

The present disclosure provides a personal care composition comprising the composition of polymer particles.

In some aspects, the personal care composition is anhydrous.

In some aspects, the personal care composition is an oil-in-water or a water-in-oil emulsion.

In some aspects, the personal care composition is a powder composition.

In some aspects, the personal care composition further comprises at least one additional component selected from the group consisting of pigments, particulates, dyes, actives, fragrances, resins, waxes, cross-linked elastomers, emulsifiers, and combinations thereof.

The present disclosure provides a personal care formulation comprising a composition of polymer particles or a personal care composition, wherein the personal care formulation is selected from the group consisting of a deodorant, an antiperspirant, a skin cream, a facial cream, a hair shampoo, a hair conditioner, a mousse, a hair styling gel, a hair spray, a protective cream, a lipstick, a facial foundation, blushes, makeup, a mascara, a skin care lotion, a moisturizer, a facial treatment, a personal cleanser, a facial cleanser, a bath oil, a perfume, a shaving cream, a pre-shave lotion, an after-shave lotion, a cologne, a sachet, and a sunscreen.

In some aspects, a composition of polymer particles is used in the manufacture of a personal care composition.

The present disclosure also provides a method of preparing a composition of polymer particles comprising:
(a) dissolving at least one terpene polymer in at least one solvent;
(b) adding a solution comprising at least one additional polymer and water,
wherein the at least one additional polymer is not a terpene polymer; and
(c) adding additional water to form polymer particles.

### BRIEF DESCRIPTION OF THE DRAWINGS

FIG. 1 shows a scanning electron microscopy (SEM) image of the poly(β-pinene) prepared by polymerization of β-pinene using the synthetic method of Example 1. The SEM image shows that the resultant poly(β-pinene) was in the form of amorphous lumps with a volume median diameter of ≥50 μm.

FIG. 2 shows a SEM image of commercially available PX 1150N poly(β-pinene) resin (Yasuhara Chemical Co., Ltd., Japan). The SEM image shows that the commercially available poly(β-pinene) resin was in the form of amorphous lumps with a volume median diameter of ≥50 μm.

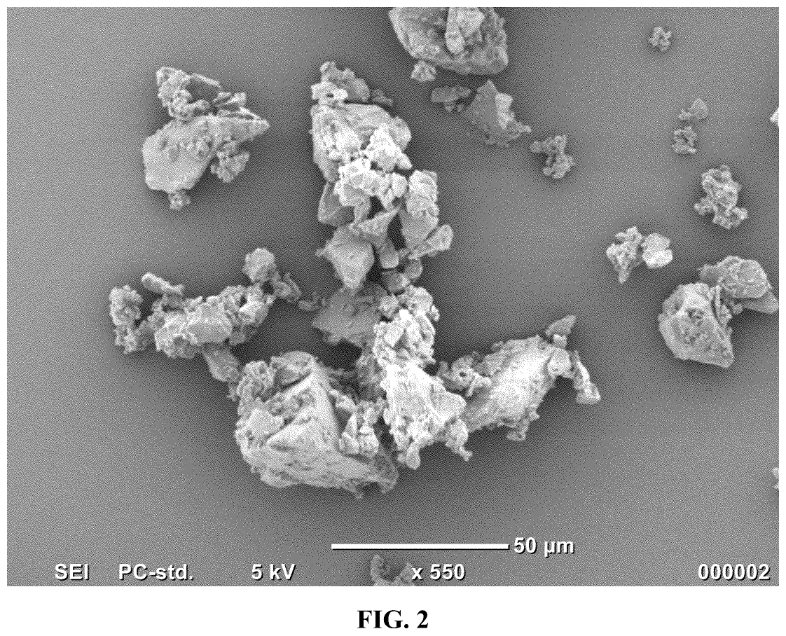

FIG. 3 shows a SEM image of the poly(β-pinene) particles prepared using the synthetic method of Example 3 and the starting poly(β-pinene) of Example 1. The SEM image shows that the poly(β-pinene) particles had a sphericity of 0.98-1 with a volume median diameter from 5 μm to 25 μm.

FIG. 4 shows a SEM image of the copolymer of poly(β-pinene) and poly(α-pinene) particles prepared using the synthetic method of Example 4 and the commercially available starting material PX 1150N poly(β-pinene) resin. The SEM image shows that the copolymer particles had a sphericity of 0.98-1 with a volume median diameter from 5 μm to 25 μm.

FIG. 5 shows a SEM image of poly(limonene) particles prepared using the synthetic method of Example 5. The SEM image shows that the poly(limonene) particles had a sphericity of 0.98-1 with a volume median diameter from 5 μm to 100 μm.

FIG. 6 shows a SEM image of hydrogenated poly(limonene) particles prepared using the synthetic method of Example 6. The SEM image shows that the hydrogenated poly(limonene) particles had a sphericity of 0.98-1 and a volume median diameter from 5 μm to 100 μm.

### DETAILED DESCRIPTION

### I. Definitions

Unless otherwise indicated, any atom with unsatisfied valences is assumed to have hydrogen atoms sufficient to satisfy the valences.

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the region of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

All ranges disclosed herein are inclusive of the endpoints, and the endpoints are independently combinable with each other. Each range disclosed herein constitutes a disclosure of any point or sub-range lying within the disclosed range. For example, the range from X to Y, is inclusive of X and Y. And, the range between X and Y, is inclusive of X and Y.

As used herein, the following definitions shall apply unless otherwise indicated. For purposes of the present disclosure, the chemical elements are identified in accordance with the Periodic Table of the Elements, CAS version, and the Handbook of Chemistry and Physics, 75^{th} Ed. 1994. Additionally, general principles of organic chemistry are described in “Organic Chemistry,” Thomas Sorrell, University Science Books, Sausalito: 1999, and “March's Advanced Organic Chemistry,” 6^{th} Ed., Smith, M. B. and March, J., eds. John Wiley & Sons, New York: 2007, the entire contents of which are hereby incorporated by reference in their entireties.

The phrase “personal care composition” as used herein, refers to a chemical mixture comprising a composition comprising polymer particles and additional components used for the purpose of cleansing, conditioning, grooming, beautifying, or otherwise enhancing the appearance of the human body.

The phrase “personal care formulation” as used herein, refers to a product marketed for use in its final form comprising a composition of polymer particles and/or a personal care composition, where the product has been further formulated for application to a human body. The personal care formulation can be in the form of a liquid, a gel, a foaming gel, a lotion, a cream, a paste, a powder, a bar, a stick, an emulsion, a dispersion, a suspension, a spray, or other commonly used forms in the industry.

Terpenes are classified using the isoprene rule (C5 rule), which is based on the number and organization of carbon atoms in the molecule, thus, grouping these molecules according to the number of isoprene units linked in a head-to-tail manner and constituting hemiterpenes (1 isoprene unit), monoterpenes (2 isoprene units; C₁₀H₁₆), sesquiterpenes (3 isoprene units; C₁₅H₂₄), and diterpenes (4 isoprene units; C₂₀H₃₂). Although the head-to-tail configuration is the most common, non-head-to-tail condensation of isoprene units also exists. The term “monomer” as used herein is an organic molecule that can be used as a raw material in a polymerization reaction, and which becomes part of a polymer backbone during the polymerization reaction.

The term “polymer” as used herein, refers to a macromolecular structure comprising repeating molecular arrangements of monomers. The term polymer is intended to encompass polymers that contain more than three repeating units.

The term “particle” as used herein, refers to a piece of matter with defined physical boundaries. The physical boundary is an interface.

The term “homopolymer” as used herein, refers to a polymer made from repeating units of a single monomer.

The term “copolymer” as used herein, refers to a polymer made from repeating units of at least two different monomers.

The term “hydrogenated” as used herein, refers to a chemical process that adds hydrogen atoms to unsaturated (double) bonds to form saturated (single) bonds. In some aspects, the extent of hydrogenation is from about 0.01% to 100%.

The weight average molecular weight (Mw) is calculated from the weight fraction distribution of different sized molecules. Since larger molecules in a sample weigh more than smaller molecules, the Mw is skewed to higher values, and is always greater than the number average molecular weight (Mₙ).

$${M}_{W} = \sum {w}_{i} {M}_{i}$$

$${M}_{n} = \sum {n}_{i} {M}_{i}$$

wherein:
wᵢ=weight fraction of chains with molecular weight Mᵢ (wᵢ=(nᵢMᵢ)/Mₙ)
nᵢ=mole fraction of chains with molecular weight Mᵢ

Various aspects of the disclosure are described in greater detail below.

### II. Compositions of Polymer Particles

The present disclosure provides improved compositions comprising polymer particles, wherein the polymer particles comprise at least one terpene polymer. The polymer particles comprising at least one terpene polymer possess a particle size sphericity and smoothness that allow these particles to blend well with other components commonly used in personal care compositions, to provide an enhanced sensory feel and to display optical benefits observable by users such as line-blurring and soft focus effect.

In some aspects, the polymer particles can comprise at least one terpene polymer, wherein the polymer particles can have a sphericity of at least 0.95 and a Dv50 particle size from about 0.5 μm to about 100 μm.

In some aspects, the polymer particles can comprise at least one terpene polymer, wherein the polymer particles have a sphericity of at least 0.95. In some aspects, the polymer particles can comprise at least one terpene polymer, wherein the polymer particles have a sphericity of at least 0.98.

In some aspects, the polymer particles can comprise at least one terpene polymer, wherein the polymer particles have a Dv50 particle size from about 0.5 μm to about 100 μm.

In some aspects, the polymer particles can comprise one terpene polymer. In some aspects, the polymer particles can comprise two terpene polymers. In some aspects, the polymer particles can comprise three terpene polymers. In some aspects, the polymer particles can comprise four terpene polymers.

In some aspects, the at least one terpene polymer is obtained by polymerizing at least one terpene monomer. In some aspects, the at least one terpene polymer is obtained by polymerizing one terpene monomer. In some aspects, the at least one terpene polymer is obtained by polymerizing two terpene monomers. In some aspects, the at least one terpene polymer is obtained by polymerizing three terpene monomers.

In some aspects, the at least one terpene monomer is selected from the group consisting of a monoterpene, a sesquiterpene, a diterpene, a sesterterpene, a triterpene, a sesquaterpene, a tetraterpene, and combinations thereof.

In some aspects, the at least one terpene polymer is obtained by polymerizing at least one monoterpene monomer. In some aspects, the at least one terpene polymer is obtained by polymerizing one monoterpene monomer. In some aspects, the at least one terpene polymer is obtained by polymerizing two monoterpene monomers. In some aspects, the at least one terpene polymer is obtained by polymerizing three monoterpene monomers.

In some aspects, the at least one terpene monomer is a monoterpene selected from the group consisting of myrcene, ocimene, alloocimene, L-limonene, D-limonene, α-pinene, β-pinene, and combinations thereof.

In some aspects, the at least one terpene polymer is a homopolymer obtained by polymerizing α-pinene, β-pinene, L-limonene, or D-limonene. In some aspects, the at least one terpene polymer is a homopolymer obtained by polymerizing β-pinene.

In some aspects, the at least one terpene polymer is a homopolymer obtained by polymerizing myrcene monomers. In some aspects, the at least one terpene polymer is obtained by polymerizing ocimene monomers. In some aspects, the at least one terpene polymer is obtained by polymerizing alloocimene monomers. In some aspects, the at least one terpene polymer is obtained by polymerizing L-limonene monomers. In some aspects, the at least one terpene polymer is obtained by polymerizing D-limonene monomers. In some aspects, the at least one terpene polymer is obtained by polymerizing α-pinene monomers. In some aspects, the at least one terpene polymer is obtained by polymerizing β-pinene monomers.

In some aspects, the at least one terpene polymer is a copolymer obtained by polymerizing α-pinene, β-pinene, L-limonene, D-limonene, or combinations thereof. In some aspects, the at least one terpene polymer is a copolymer obtained by polymerizing α-pinene monomers and β-pinene monomers. In some aspects, the at least one terpene polymer is a copolymer obtained by polymerizing α-pinene monomers and L-limonene monomers. In some aspects, the at least one terpene polymer is a copolymer obtained by polymerizing α-pinene monomers and D-limonene monomers. In some aspects, the at least one terpene polymer is a copolymer obtained by polymerizing β-pinene monomers and D-limonene monomers. In some aspects, the at least one terpene polymer is a copolymer obtained by polymerizing β-pinene monomers and L-limonene monomers. In some aspects, the at least one terpene polymer is a copolymer obtained by polymerizing D-limonene and L-limonene monomers.

In some aspects, the at least one terpene polymer is a homopolymer obtained by polymerizing at least one hydrogenated terpene monomer. In some aspects, the at least one terpene polymer is a homopolymer obtained by hydrogenating a terpene monomer selected from the group consisting of myrcene, ocimene, alloocimene, L-limonene, D-limonene, α-pinene, and β-pinene. In some aspects, the at least one terpene polymer is a homopolymer obtained by polymerizing hydrogenated α-pinene. In some aspects, the at least one terpene polymer is a homopolymer obtained by polymerizing hydrogenated β-pinene. In some aspects, the at least one terpene polymer is a homopolymer obtained by polymerizing hydrogenated limonene. In some aspects, the at least one terpene polymer is a homopolymer obtained by polymerizing hydrogenated D-limonene. In some aspects, the at least one terpene polymer is a homopolymer obtained by polymerizing hydrogenated L-limonene.

In some aspects, the at least one terpene monomer is hydrogenated from about 0.1% to 100%. In some aspects, the at least one terpene monomer is hydrogenated from about 0.1% to 100%, from about 0.1% to about 99%, from about 0.1% to about 90%, from about 0.1% to about 75%, from about 0.1% to about 50%, from about 0.1% to about 25%, from about 0.1% to about 10%, from about 0.1% to about 1%, from about 1% to 100%, from about 1% to about 99%, from about 1% to about 90%, from about 1% to about 75%, from about 1% to about 50%, from about 1% to about 25%, from about 1% to about 10%, from about 10% to 100%, from about 10% to about 99%, from about 10% to about 90%, from about 10% to about 75%, from about 10% to about 50%, from about 10% to about 25%, from about 25% to 100%, from about 25% to about 99%, from about 25% to about 90%, from about 25% to about 75%, from about 25% to about 50%, from about 50% to 100%, from about 50% to about 99%, from about 50% to about 90%, from about 50% to about 75%, from about 75% to 100%, from about 75% to about 99%, from about 75% to about 90%, from about 90% to 100%, from about 90% to about 99%, or from about 99% to 100%.

In some aspects, the at least one terpene polymer is a homopolymer obtained by hydrogenating at least one terpene polymer. In some aspects, the at least one terpene polymer is a homopolymer obtained by hydrogenating a terpene polymer selected from the group consisting of poly(myrcene), poly(ocimene), poly(alloocimene), poly(L-limonene), poly(D-limonene), poly(α-pinene), and poly(β-pinene). In some aspects, the at least one terpene polymer is a homopolymer obtained by hydrogenating poly(α-pinene). In some aspects, the at least one terpene polymer is a homopolymer obtained by hydrogenating poly(β-pinene). In some aspects, the at least one terpene polymer is a homopolymer obtained by hydrogenating poly(limonene). In some aspects, the at least one terpene polymer is a homopolymer obtained by hydrogenating poly(D-limonene). In some aspects, the at least one terpene polymer is a homopolymer obtained by hydrogenating poly(L-limonene).

In some aspects, the at least one terpene polymer is hydrogenated from about 0.1% to 100%. In some aspects, the at least one terpene polymer is hydrogenated from about 0.1% to about 100%, from about 0.1% to about 99%, from about 0.1% to about 90%, from about 0.1% to about 75%, from about 0.1% to about 50%, from about 0.1% to about 25%, from about 0.1% to about 10%, from about 0.1% to about 1%, from about 1% to 100%, from about 1% to about 99%, from about 1% to about 90%, from about 1% to about 75%, from about 1% to about 50%, from about 1% to about 25%, from about 1% to about 10%, from about 10% to 100%, from about 10% to about 99%, from about 10% to about 90%, from about 10% to about 75%, from about 10% to about 50%, from about 10% to about 25%, from about 25% to 100%, from about 25% to about 99%, from about 25% to about 90%, from about 25% to about 75%, from about 25% to about 50%, from about 50% to 100%, from about 50% to about 99%, from about 50% to about 90%, from about 50% to about 75%, from about 75% to 100%, from about 75% to about 99%, from about 75% to about 90%, from about 90% to 100%, from about 90% to about 99%, or from about 99% to 100%.

In some aspects, the at least one terpene polymer is a copolymer obtained by hydrogenating a copolymer of at least two terpene polymers. In some aspects, the at least one terpene polymer is obtained by hydrogenating a copolymer of at least one terpene monomer and at least one monomer that is not a terpene.

In some aspects, the at least one terpene polymer is a copolymer further comprising at least one monomer that is not a terpene or the hydrogenation product of a terpene.

In some aspects, the at least one monomer that is not a terpene is selected from the group consisting of an acrylate, an anhydride, a styrene, an ethylene, a butadiene, a urethane, a propylene, an imidazole, a silicone, a vinyl pyridine, a carbonate, and combinations thereof.

In some aspects, the weight average molecular weight (Mw) of the at least one terpene polymer is greater than or equal to about 200 g/mol. In some aspects, the weight average molecular weight of the at least one terpene polymer is from about 200 g/mol to about 2,000,000 g/mol. In some aspects, the weight average molecular weight of the at least one terpene polymer is from about 200 g/mol to about 20,000 g/mol. In some aspects, the weight average molecular weight of the at least one terpene polymer is from about 200 g/mol to about 10,000 g/mol. In some aspects, the weight average molecular weight of the at least one terpene polymer is from about 200 g/mol to about 2,000,000 g/mol, from about 200 g/mol to about 1,000,000 g/mol, from about 200 g/mol to about 500,000 g/mol, from about 200 g/mol to about 100,000 g/mol, from about 200 g/mol to about 20,000 g/mol, from about 200 g/mol to about 10,000 g/mol, from about 200 g/mol to about 5,000 g/mol, from about 200 g/mol to about 2,500 g/mol, from about 200 g/mol to about 1,000 g/mol, from about 200 g/mol to about 500 g/mol, from about 500 g/mol to about 2,000,000 g/mol, from about 500 g/mol to about 1,000,000 g/mol, from about 500 g/mol to about 500,000 g/mol, from about 500 g/mol to about 100,000 g/mol, from about 500 g/mol to about 20,000 g/mol, from about 500 g/mol to about 10,000 g/mol, from about 500 g/mol to about 5,000 g/mol, from about 500 g/mol to about 2,500 g/mol, from about 500 g/mol to about 1,000 g/mol, from about 1,000 g/mol to about 2,000,000 g/mol, from about 1,000 g/mol to about 1,000,000 g/mol, from about 1,000 g/mol to about 500,000 g/mol, from about 1,000 g/mol to about 100,000 g/mol, from about 1,000 g/mol to about 20,000 g/mol, from about 1,000 g/mol to about 10,000 g/mol, from about 1,000 g/mol to about 5,000 g/mol, from about 1,000 g/mol to about 2,500 g/mol, from about 2,500 g/mol to about 2,000,000 g/mol, from about 2,500 g/mol to about 1,000,000 g/mol, from about 2,500 g/mol to about 500,000 g/mol, from about 2,500 g/mol to about 100,000 g/mol, from about 2,500 g/mol to about 20,000 g/mol, from about 2,500 g/mol to about 10,000 g/mol, from about 2,500 g/mol to about 5,000 g/mol, from about 5,000 g/mol to about 2,000,000 g/mol, from about 5,000 g/mol to about 1,000,000 g/mol, from about 5,000 g/mol to about 500,000 g/mol, from about 5,000 g/mol to about 100,000 g/mol, from about 5,000 g/mol to about 20,000 g/mol, from about 5,000 g/mol to about 10,000 g/mol, from about 10,000 g/mol to about 2,000,000 g/mol, from about 10,000 g/mol to about 1,000,000 g/mol, from about 10,000 g/mol to about 500,000 g/mol, from about 10,000 g/mol to about 100,000 g/mol, from about 10,000 g/mol to about 20,000 g/mol, from about 20,000 g/mol to about 2,000,000 g/mol, from about 20,000 g/mol to about 1,000,000 g/mol, from about 20,000 g/mol to about 500,000 g/mol, from about 20,000 g/mol to about 100,000 g/mol, from about 100,000 g/mol to about 2,000,000 g/mol, from about 100,000 g/mol to about 1,000,000 g/mol, from about 100,000 g/mol to about 500,000 g/mol, from about 500,000 g/mol to about 2,000,000 g/mol, from about 500,000 g/mol to about 1,000,000 g/mol, or from about 1,000,000 g/mol to about 2,000,000 g/mol.

In some aspects, the weight average molecular weight of the at least one terpene polymer is about 200 g/mol, about 500 g/mol, about 1,000 g/mol, about 2,500 g/mol, about 5,000 g/mol, about 10,000 g/mol, about 25,000 g/mol, about 50,000 g/mol, about 75,000 g/mol, about 100,000 g/mol, about 250,000 g/mol, about 500,000 g/mol, about 750,000 g/mol, about 1,000,000 g/mol, about 1,250,000 g/mol, about 1,500,000 g/mol, 1,750,000 g/mol, or about 2,000,000 g/mol. In some aspects, the weight average molecular weight of the at least one terpene polymer is about 10,000 g/mol. In some aspects, the weight average molecular weight of the at least one terpene polymer is about 20,000 g/mol. The weight average molecular weight is measured by gel permeation chromatography.

The softening point of a terpene polymer is the temperature at which the polymer flows. Softening point can be measured by Differential Scanning calorimetry (DSC) using a DSC 250 (TA Instruments, New Castle, DE). The onset of the glass transition is indicated by the softening of the polymer and ends once the polymer is completely free-flowing. Softening point is assigned to the temperature at the peak of the heat flow curve obtained by DSC.

In some aspects, the terpene polymer can have a softening point of greater than or equal to about 50° C. In some aspects, the terpene polymer can have a softening point of greater than or equal to about 80° C. In some aspects, the terpene polymer can have a softening point of from about 50° C. to about 300° C., from about 50° to about 200° C., from about 50° C. to about 100° C., from about 50° C. to about 80° C., from about 80° C. to about 300° C., from about 80° C. to about 200° C., from about 80° C. to about 100° C., from about 100° C. to about 300° C., from about 100° C. to about 200° C., or from about 200° C. to about 300° C.

Sphericity, S, is defined as the degree to which the particle is similar to a sphere. The sphericity of a particle can be measured by the following method. Using a JCM-6000 benchtop Scanning Electron Microscope (SEM) manufactured by JEOL Ltd, Japan, an image of particles can be taken and the major axis and minor axis of 30 randomly selected particles can be measured. The major axis is the line segment going through the farthest points on a particle. The minor axis is the segment going through the closest. Then a minor axis/major axis ratio of each particle can be determined. The average value of the minor axis/major axis ratio for the 30 particles can be calculated to provide the average sphericity of the particles. The sphericity of a sphere is 1 and any particle which is not a sphere will have sphericity of less than 1. A particle is considered to be spherical if its sphericity falls within the range of about 0.98 to 1.

In some aspects, the polymer particles in the composition can have a sphericity from about 0.90 to about 1.0 as measured by SEM. In some aspects, the polymer particles in the composition can have a sphericity from about 0.95 to about 1.0. In some aspects, the polymer particles in the composition can have a sphericity from about 0.97 to about 1.0. In some aspects, the polymer particles in the composition can have a sphericity from about 0.98 to about 1. In some aspects, the polymer particles in the composition can have a sphericity of about 0.99. In some aspects, the polymer particles in the composition can have a sphericity of about 0.90, about 0.91, about 0.92, about 0.93, about 0.94, about 0.95, about 0.96, about 0.97, about 0.98, about 0.99, or about 1.0.

Smoothness is defined as a quality of particle surface being smooth. A smooth particle is a particle that has an even and regular surface, free of perceptible lumps or indentations. One way to measure smoothness is to measure gradient in greyscale value of pixels for a line segment drawn on particle surface. Greyscale value is define as 0 means black, 255 means white. In between, every other number is a shade of gray ranging from black to white. Smoothness of a particle can be quantified using from SEM images of particles using a software ImageJ. A line is drawn across a particle and the intensity profile is plotted. Absence of sharp peaks and/or presence of small peaks with the gray value delta of <20 is an indication of a smooth surface of the particle. Sharp peaks and/or peaks of the gray value delta of >20, are an indication of rough surface of a particle.

In some aspects, the polymer particles in the composition can have a smoothness from about 1 to about 20, from about 1 to about 15, from about 1 to about 10, from about 1 to about 5, from about 5 to about 20, from about 5 to about 15, from about 5 to about 10, from about 10 to about 20, from about 10 to about 15, or from about 15 to about 20. In some aspects, the polymer particles in the composition can have a smoothness of about 1, about 5, about 10, about 15, or about 20.

Porosity is defined as the percent of voids within a volume of a shape and is an indication of how uniform and homogenous a shape is. Porosity can be measured using a JCM-6000 benchtop Scanning Electron Microscope (SEM) manufactured by JEOL Ltd, Japan. 30 random particles can be assessed for the presence/absence of recessed areas using SEM. The recessed area is identified as any black colored area observed in SEM image of particles. Particles with no visible recessed areas are classified as nonporous. Particles with visible recessed areas are classified as porous.

Hollowness is defined as the quality of having empty cavity inside and is the opposite of solidity, which is defined as the state of having the interior filled with matter. Solidity can be quantified using a JCM-6000 benchtop Scanning Electron Microscope (SEM) manufactured by JEOL Ltd, Japan. First, particles can be fractured by grinding them with a mortar and pestle. Then an SEM image of ground particles can be taken. 30 random particles can then be assessed for presence of matter inside the broken shell. Particles can be classified either hollow or solid. Presence of empty cavity can be confirmed by SEM images. The hollow area/cavity is identified as black/grey colored area observed in SEM image of particles which is enclosed by shell. Thickness of the shell can be measured using the Scaler option in the SEM software.

A particle size distribution can be expressed using four values. “D”, represents the percentage of the particles in the composition that are smaller than an indicated size. The analyzer used with laser diffraction to determine particle size does not measure the particles one by one, but rather uses light with different angles and then retrieves the diffraction patterns from image sensors. Then, by performing addition, subtraction, and cross-analysis calculations, the instrument determines the statistical proportion of the sizes of the particles.

The instrument also calculates the volume median diameter using software. The volume median diameter (Dv50) is the particle diameter for which 50% of the particle volume is contained in smaller particle diameter than Dv50 and 50% is contained in larger particle diameter than Dv50. Therefore, the Dv50 is half the total particle volume. Particle size distribution of the polymer particles can be measured using the following method. Polymer particles were suspended in cyclopentasiloxane and sonicated for 10 minutes. A cuvette was filled with cyclopentasiloxane and placed in the particle size analyzer, LA-960 laser diffraction particle size analyzer manufactured by Horiba Scientific, Japan. A few drops of the suspension containing polymer particles can then be transferred into the cuvette with cyclopentasiloxane and stirred with a magnet. Once the transmittance reached the acceptable range, the measurement can be performed.

In some aspects, the polymer particles in the composition can have a Dv50 particle size from about 0.5 μm to about 100 μm. In some aspects, the polymer particles in the composition have a Dv50 particle size from about 0.5 μm to about 25 μm. In some aspects, the polymer particles in the composition have a Dv50 particle size from about 5 μm to about 15 μm. In some aspects, the polymer particles in the composition can have a Dv50 particle size from about 0.5 μm to about 100 μm, from about 0.5 μm to about 80 μm, from about 0.5 μm to about 60 μm, from about 0.5 μm to about 40 μm, from about 0.5 μm to about 30 μm, from about 0.5 μm to about 20 μm, from about 0.5 μm to about 10 μm, from about 0.5 μm to about 5 μm, from about 0.5 μm to about 1 μm, from about 1 μm to about 100 μm, from about 1 μm to about 80 μm, from about 1 μm to about 60 μm, from about 1 μm to about 40 μm, from about 1 μm to about 30 μm, from about 1 μm to about 20 μm, from about 1 μm to about 10 μm, from about 1 μm to about 5 μm, from about 5 μm to about 100 μm, from about 5 μm to about 80 μm, from about 5 μm to about 60 μm, from about 5 μm to about 40 μm, from about 5 μm to about 30 μm, from about 5 μm to about 20 μm, from about 5 μm to about 10 μm, from about 10 μm to about 100 μm, from about 10 μm to about 80 μm, from about 10 μm to about 60 μm, from about 10 μm to about 40 μm, from about 10 μm to about 30 μm, from about 10 μm to about 20 μm, from about 20 μm to about 100 μm, from about 20 μm to about 80 μm, from about 20 μm to about 60 μm, from about 20 μm to about 40 μm, from about 20 μm to about 30 μm, from about 30 μm to about 100 μm, from about 30 μm to about 80 μm, from about 30 μm to about 60 μm, from about 30 μm to about 40 μm, from about 40 μm to about 100 μm, from about 40 μm to about 80 μm, from about 40 μm to about 60 μm, from about 60 μm to about 100 μm, from about 60 μm to about 80 μm, or from about 80 μm to about 100 μm, In some aspects, the polymer particles in the composition can have a Dv50 particle size of about 20 μm.

In some aspects, the composition of polymer particles are insoluble in non-polar oils. As used herein, the phrase “non-polar oils” are oils which are lacking an electronegative element such as oxygen. The measure of polarity is the dielectric constant. The higher the dielectric constant of a solvent, the more polar it is. In some aspects, the non-polar oil is selected from the group consisting of a hydrocarbon, a silicone oil, a vegetable oil, a mineral oil, and combinations thereof. In some aspects, the non-polar oil is selected from the group consisting of dodecane, isododecane, tetradecane, isohexadecane, castor oil, flaxseed oil, silicone oil, and combinations thereof.

### III Methods of Preparing a Composition of Polymer Particles

In some aspects, the present disclosure provides methods of preparing a composition of polymer particles comprising:
(a) dissolving at least one terpene polymer in at least one solvent;
(b) adding a solution comprising at least one additional polymer and water, wherein the at least one additional polymer is not a terpene polymer; and
(c) adding additional water to form polymer particles.

In some aspects, the methods of preparing a composition of polymer particles comprise dissolving one terpene polymer. In some aspects, the methods of preparing a composition of polymer particles comprise dissolving two terpene polymers. In some aspects, the methods of preparing a composition comprising polymer particles comprise dissolving three terpene polymers.

In some aspects, the methods of preparing a composition of polymer particles comprises at least one additional polymer that is not a terpene polymer. In some aspects, the methods of preparing a composition of polymer particles comprises one additional polymer. In some aspects, the methods of preparing a composition of polymer particles comprises two additional polymers. In some aspects, the methods of preparing a composition of polymer particles comprises three additional polymers.

In some aspects, the at least one solvent is selected from the group consisting of butane, propane, hexane, isopropyl alcohol, ethanol, ethyl acetate, acetone, methanol, chloroform, methylene chloride, and combinations thereof.

In some aspects, the molar ratio of the total moles of the terpene polymer(s) to the total moles of the solvent(s) is from about 1:4 to about 2:3. In some aspects, the molar ratio of the total moles of the terpene polymer(s) to the total moles of the solvent(s) is from about 1:4 to about 2:3, from about 1:4 to about 1:2.5, from about 1:4 to about 1:1.5, from about 1:4 to about 1:1, from about 1:1 to about 1:4, from about 1:1 to about 1:1.5, or from about 1:1 to about 1:2.5. In some aspects, the molar ratio of the total moles of the terpene polymer(s) to the total moles of the solvent(s) is from about 1:4, about 1:3, about 1:2.5, about 1:1.5, or about 2:3.

In some aspects, the at least one solvent can be ethyl acetate. In some aspects, the molar ratio of the total moles of the terpene polymer(s) to the total moles of ethyl acetate is from about 1:3 to about 1:1.5. In some aspects, the molar ratio of the total moles of the terpene polymer(s) to the total moles of ethyl acetate is from about 1:2.5 to about 1:1.5. In some aspects, the molar ratio of the total moles of the terpene polymer(s) to the total moles of ethyl acetate is from about 1:4, about 1:3, about 1:2.5, about 1:1.5, or about 2:3.

In some aspects, the at least one solvent can be methylene chloride. In some aspects, the molar ratio of the total moles of the terpene polymer(s) to the total moles of methylene chloride is from about 1:3 to about 1:1.5. In some aspects, the molar ratio of the total moles of the terpene polymer(s) to the total moles of methylene chloride is from about 1:2.5 to about 1:1.5. In some aspects, the molar ratio of the total moles the terpene polymer(s) to the total moles of methylene chloride is from about 1:4, about 1:3, about 1:2.5, about 1:1.5, or about 2:3.

In some aspects, the molar ratio of the total moles of the terpene polymer(s) to the total moles of the additional polymer(s) is from about 1:4 to about 2:3. In some aspects, the at least one additional polymer can be PVA. In some aspects, the molar ratio of the total moles of the terpene polymer(s) to the total moles of PVA is from about 1:3 to about 1:1.5. In some aspects, the molar ratio of the total moles the terpene polymer(s) to the total moles of PVA is from about 1:2.5 to about 1:1.5. In some aspects, the molar ratio of the total moles of the terpene polymer(s) to the total moles of PVA is from about 1:4, about 1:3, about 1:2.5, about 1:1.5, or about 2:3.

In some aspects, the mixing time for preparing a composition of polymer particles can occur at a speed from about 5 rpm to about 2000 rpm. In some aspects, the mixing can occur at a speed from about 10 rpm to about 900 rpm. In some aspects, the mixing can occur at a speed from about 10 rpm to about 800 rpm. In some aspects, the mixing can occur at a speed from about 50 rpm to about 700 rpm. In some aspects, the mixing can occur at a speed of about 5 rpm, about 10 rpm, about 15 rpm, about 20 rpm, about 25 rpm, about 30 rpm, about 35 rpm, about 40 rpm, about 45 rpm, about 50 rpm, about 55 rpm, about 60 rpm, about 65 rpm, about 70 rpm, about 75 rpm, about 80 rpm, about 85 rpm, about 90 rpm, about 95 rpm, about 100 rpm, about 150 rpm, about 200 rpm, about 250 rpm, about 300 rpm, about 350 rpm, about 400 rpm, about 450 rpm, about 500 rpm, about 550 rpm, about 600 rpm, about 650 rpm, about 700 rpm, about 750 rpm, about 800 rpm, about 850 rpm, about 900 rpm, about 950 rpm, or about 1000 rpm. In some aspects, the mixing can occur at a speed of about 600 rpm. In some aspects, the mixing can occur at a speed of about 700 rpm. In some aspects, the mixing can occur at a speed of about 900 rpm.

### IV. Personal Care Composition and Personal Care Formulation

In some aspects, a personal care composition comprising a composition of polymer particles can further comprise at least one additional component selected from the group consisting of water, aqueous solvent (e.g. alcohol or other water compatible solvent), non-aqueous solvent, emollients, humectants, oils (polar and non-polar), conditioning agents, emulsifiers, surfactants, thickeners, stiffening agents, medicaments, fragrances, preservatives, deodorant components, antiperspirant compounds, skin protecting agents, pigments, particulates, dyes, sunscreens, and combinations thereof.

In some aspects, a composition of polymer particles is used in the manufacture of a personal care composition.

In some aspects, a personal care composition comprising a composition of polymer particles can further comprise at least one additional component selected from the group consisting of pigments, particulates, dyes, actives, fragrances, resins, waxes, cross-linked elastomers, emulsifiers, and combinations thereof.

In some aspects, a personal care composition comprising a composition of polymer particles can further comprise at least one additional component selected from the group consisting of pigments, particulates, dyes, and combinations thereof.

In some aspects, the pigments, particulates, and dyes are selected from the group consisting of iron oxides, zinc oxides, titanium dioxides, mica, silica, tapioca starch, rice starch, cellulose, cellulose acetate, polymethylsilsesquioxane, mica, and combinations thereof.

In some aspects, the total weight of the additional component(s) can comprise from about 0.01% to about 80%, from about 0.01% to about 60%, from about 0.01% to about 40%, from about 0.01% to about 20%, from about 0.01% to about 10%, from about 0.01% to about 5%, from about 0.01% to about 1%, from about 1% to about 80%, from about 1% to about 60%, from about 1% to about 40%, from about 1% to about 20%, from about 1% to about 10%, from about 1% to about 5%, from about 5% to about 80%, from about 5% to about 60%, from about 5% to about 40%, from about 5% to about 20%, from about 5% to about 10%, from about 10% to about 80%, from about 10% to about 60%, from about 10% to about 40%, from about 10% to about 20%, from about 20% to about 80%, from about 20% to about 60%, from about 20% to about 40%, from about 40% to about 80%, from about 40% to about 60%, or from about 60% to about 80% of the total weight of the composition. In some aspects, the personal care compositions can comprise a total weight of the additional component(s) in a weight ratio of about 0.01%, about 1%, about 5%, about 10%, about 20%, about 30%, about 40%, about 50%, about 60%, about 70%, or about 80% of the total weight of the personal care composition.

In some aspects, a personal care formulation can be prepared by combining a personal care composition or a composition comprising polymer particles with at least one solvent to form a solid, gel, a paste, or a powder. In some aspects, the solvent is selected from the group consisting of water, an alcohol, a ketone, an ether, and an ester.

In some aspects, the personal care formulation is a solid. In some aspects, the personal care formulation is a gel. In some aspects, the personal care formulation is a paste. In some aspects, the personal care formulation is a powder.

In some aspects, a personal care formulation can be prepared by shearing the composition comprising polymer particles, as described herein, with a solvent, as described herein, to form a sheared gel. In some aspects, a personal care formulation can be prepared by combining a personal care composition or a composition comprising polymer particles, as described herein, with a solvent thereby forming a mixture and shearing the mixture.

In some aspects, the personal care formulation comprising polymer particles can be crumbled to form a powder.

In some aspects, the composition is in the form of an emulsion.

As used herein, the term “emulsion” refers to a colloidal dispersion of two or more liquid immiscible phases (or substantially immiscible phases) in the form of droplets. One of the liquid phases is normally a dispersed phase and another one is a continuous phase, wherein the dispersed phase is scattered in the continuous phase as a plurality of droplets. The emulsion can be in a form of a macroemulsion, a microemulsion, or a nanoemulsion based on the size of the droplets. The emulsion is an oil-in-water (o/w) emulsion if the continuous phase is an aqueous solution or a water-in-oil (w/o)-type if the continuous phase is an oil. Other examples of emulsions include oil-in-water-in-oil (o/w/o) emulsions, which comprise oil droplets contained within aqueous droplets dispersed in a continuous oil phase. As used herein the term “oil” refers to any nonpolar chemical substance that is in liquid form at ambient temperature and atmospheric pressure and is both hydrophobic and lipophilic.

In some aspects, the composition is in the form of a microemulsion. A microemulsion has a droplet size in micrometers (10⁻⁶).

In some aspects, the composition is in the form of a nanoemulsion. A nanoemulsion has a droplet size in nanometers (10⁻⁹).

In some embodiments, at least about 60% of the at least one compound of formula (I) is present in a dispersed phase. In some aspects, at least about 70% of the personal care composition is in the dispersed phase. In some aspects, at least about 80% of the personal care composition is in the dispersed phase. In some aspects, at least about 90% of the personal care composition is in the dispersed phase. In some aspects, at least about 95% of the personal care composition is in the dispersed phase.

In some aspects, between about 60% and about 95% of the personal care composition is in the dispersed phase. In some aspects, the percentage of the personal care composition in the dispersed phase is between about 60% and about 95%, about 60% and about 90%, about 60% and about 80% about 60% and about 70%, about 70% and about 95%, about 70% and about 90%, about 70% and about 80%, about 80% and about 95%, about 80% and about 90%, or about 90% and about 95%.

In some aspects, the personal care composition is anhydrous. As used herein, the term “anhydrous” means that there is less than 2% by weight of water added to a composition based on the total weight of the composition. In some aspects, the compositions can comprise less than about 2% by weight of water, less than about 1% by weight of water, less than about 0.5% by weight of water, or less than 0.4% by weight of water.

In some aspects, the personal care formulation is for application to a keratinous substrate, to a fabric, or to leather.

In some aspects, the personal care formulation is selected from the group consisting of a deodorant, an antiperspirant, a skin cream, a facial cream, a hair shampoo, a hair conditioner, a mousse, a hair styling gel, a hair spray, a protective cream, a lipstick, a facial foundation, blushes, makeup, a mascara, a skin care lotion, a moisturizer, a facial treatment, a personal cleanser, a facial cleanser, a bath oil, a perfume, a shaving cream, a pre-shave lotion, an after-shave lotion, a cologne, a sachet, and a sunscreen.

### V. Composition of Polymer Particles Prepared by Process

The present disclosure provides a composition comprising polymer particles, wherein the polymer particles are prepared by (a) dissolving at least one terpene polymer in at least one solvent; (b) adding a solution comprising at least one additional polymer and water, wherein the at least one additional polymer is not a terpene polymer; and (c) adding additional water to form polymer particles, wherein the at least one terpene polymer, the at least one solvent, and the at least one additional polymer are defined herein.

### EXAMPLES

The following examples are included to demonstrate various aspects of the present disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific examples which are disclosed and still obtain a like or similar result without departing from the spirit and scope of the disclosure.

### Measurement Techniques Used in the Examples:

Solidity was measured using a JCM-6000 benchtop Scanning Electron Microscope (SEM) manufactured by JEOL Ltd, Japan. First, particles were fractured by grinding them with a mortar and pestle. Then an SEM image of ground particles was taken. 30 random particles were assessed for the presence/absence of recessed areas using SEM. The recessed area was identified as any black colored area observed in the SEM image of the ground particles. Particles with no visible recessed areas were classified as solid and were given a solidity value(S) of 100%.

Sphericity was measured using a JCM-6000 benchtop Scanning Electron Microscope (SEM) manufactured by JEOL Ltd, Japan. An image of particles was taken and the major axis and minor axis of 30 randomly selected particles was measured. The major axis is the line segment going through the farthest points on a particle. The minor axis is the segment going through the closest. Then a minor axis/major axis ratio of each particle was determined. The average value of the minor axis/major axis ratio for the 30 particles was calculated to provide the average sphericity of the particles.

Particle size distribution of the polymer particles was measured by suspending the polymer particles in cyclopentasiloxane and sonicating for 10 minutes. A cuvette was filled with cyclopentasiloxane and placed in the particle size analyzer, LA-960 laser diffraction particle size analyzer manufactured by Horiba Scientific, Japan. A few drops of the suspension containing polymer particles was then transferred into the cuvette with cyclopentasiloxane and stirred with a magnet. Once the transmittance reached the acceptable range, the measurement was performed.

### Example 1: Preparation of Poly(β-pinene)

An AlCl₃OPh₂ complex was prepared by adding 3.6 mL (0.021 mol) Ph₂O dropwise to a slurry of 3 g (0.022 mol) AlCl₃ in 18.7 mL of CH₂Cl₂ over 5-10 minutes. The mixture was allowed to stir for 30-60 minutes to provide complete dissolution of the AlCl₃ and provide the AlCl₃OPh₂ complex in a solution with CH₂Cl₂.

Polymerization was initiated by adding 0.07 mL (1 M) of the AlCl₃OPh₂ complex in a solution with CH₂Cl₂ to a mixture of 1.76 mL (0.0149 M) of β-pinene, 15 mL of CH₂Cl₂, and 10 mL of n-hexane. 3-5 mL aliquots were withdrawn from the mixture and poured into ethanol to determine the reaction progress. The precipitated polymer was separated from the mixture by centrifugation and the resultant polymer was dried in vacuum.

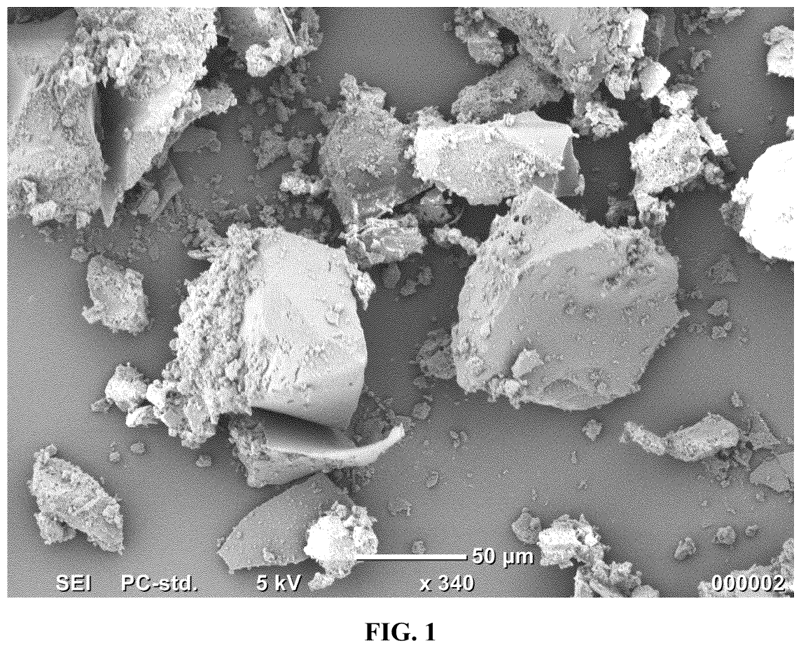

The resultant poly(β-pinene) materials are shown in FIG. 1, have a sphericity of less than 0.5, a solidity of less than 50%, and are in large chunks with a volume median diameter particle size of greater than or equal to 50 μm.

### Example 2: Preparation of a PVA Stock Solution

A polyvinyl alcohol (PVA) stock solution (1:10 parts PVA:deionized water) was prepared by adding 1 part PVA to 10 parts deionized water at room temperature while stirring for approximately 30 minutes. The temperature was increased to approximately 70° C. with stirring for 30 minutes to dissolve the PVA. The solution was removed from heat and allowed to cool to room temperature with stirring. The stock solution was stored at room temperature.

To prepare a diluted PVA stock solution: added 90 grams of deionized water to 30 grams of the PVA stock solution and mixed well.

### Example 3: Preparation of Poly(β-pinene) Particles

40 grams of the diluted PVA stock solution of Example 2 was added to an 800 mL plastic beaker equipped with a Cowles blade rotating at 500 rpm. 40 grams of the 10% poly(β-pinene) of Example 1 dissolved in ethyl acetate was added to the beaker under stirring at 500 rpm. Stirring was continued for 10 minutes. 400 mL of deionized water was further slowly added to the beaker after initial 10 minutes. After 20 minutes, the mixture was transferred to a 2 L glass beaker. 1 L of deionized water was added to the mixture over 10 minutes at a stirring speed of 500 rpm. The mixture was stirred at 500 rpm for 2 hours.

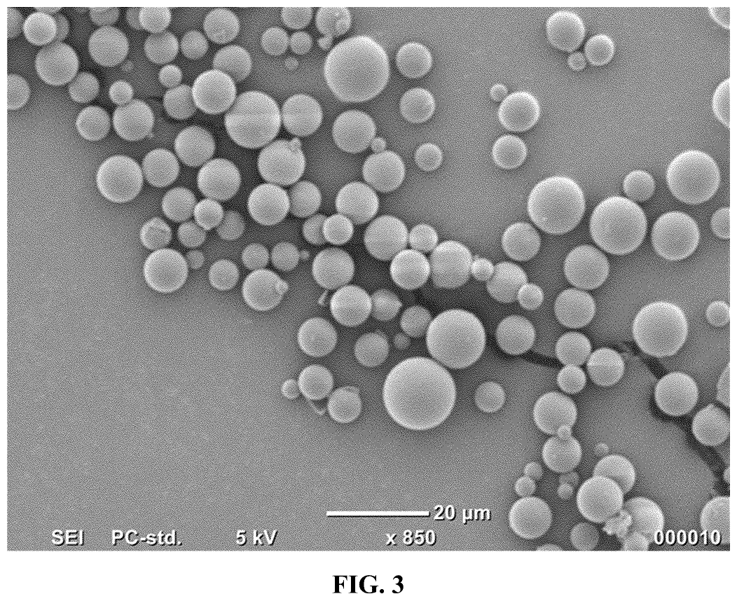

The resultant poly(β-pinene) particles, shown in FIG. 3, have a sphericity of 1, they are non-porous and have a volume median diameter particle size from about 5 μm to about 25 μm.

### Example 4: Preparation of Mixed Pinene Copolymer Particles

20 grams of the diluted PVA stock solution of Example 2 was added to an 800 mL plastic beaker equipped with a Cowles blade rotating at 500 rpm. 40 grams of a 10% commercially available terpene copolymer of α-pinene and β-pinene monomers (YS Resin PX 1150N, Yasuhara Chemical Co., Ltd., Japan (yellow solid with a softening point of 100-110° C.)) dissolved in CH₂Cl₂ was added to the beaker under stirring at 500 rpm. Stirring was continued for 10 minutes. 80 mL of deionized water was further added to the beaker after initial 10 minutes. The mixture was then transferred to a 2 L glass beaker. 1 L of deionized water was added to the mixture over 10 minutes at a stirring speed of 500 rpm. An additional 1.5 L of deionized water was added instantly to the mixture and stirring is continued at 500 rpm for 2 hours.

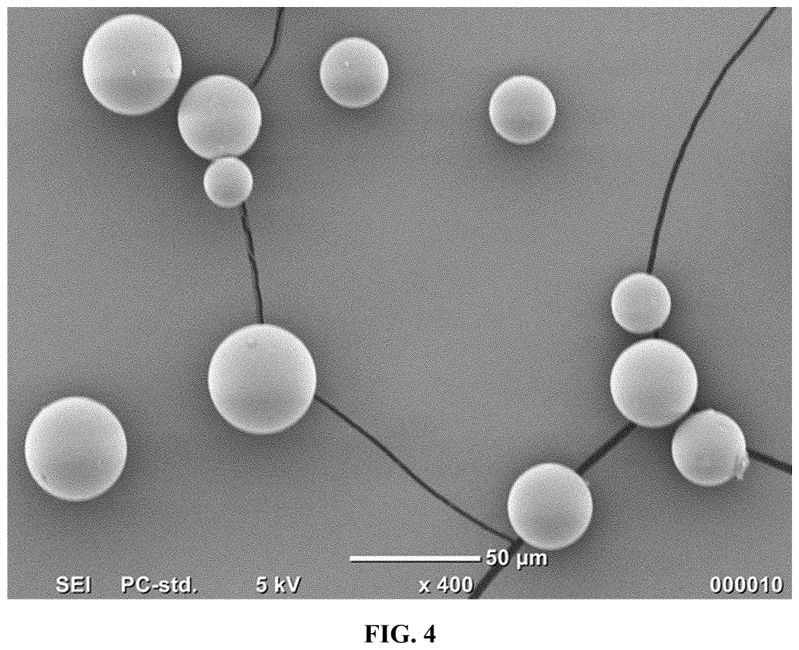

The resultant mixed pinene copolymer particles are shown in FIG. 4, have a sphericity of 1, are non-porous, and have a volume median diameter particle size from about 5 μm to about 25 μm.

### Example 5: Preparation of Poly(limonene) Particles

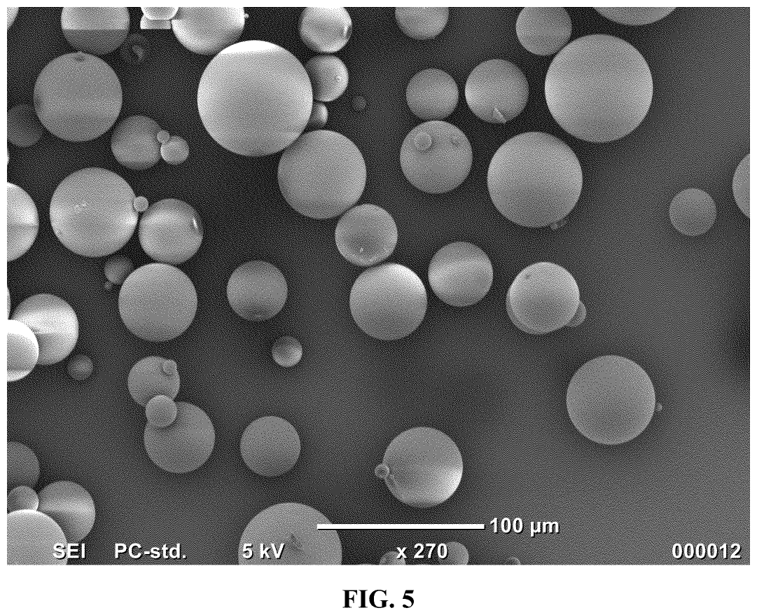

Poly(limonene) was obtained using the synthetic method disclosed in Lyubushkin, A., et al., ChemEngineering 7 (1): 11 pages (2023). Starting with poly(limonene), the method of Example 3 was followed to obtain the poly(limonene) particles shown in FIG. 5.

### Example 6: Preparation of Hydrogenated Poly(limonene) Particles

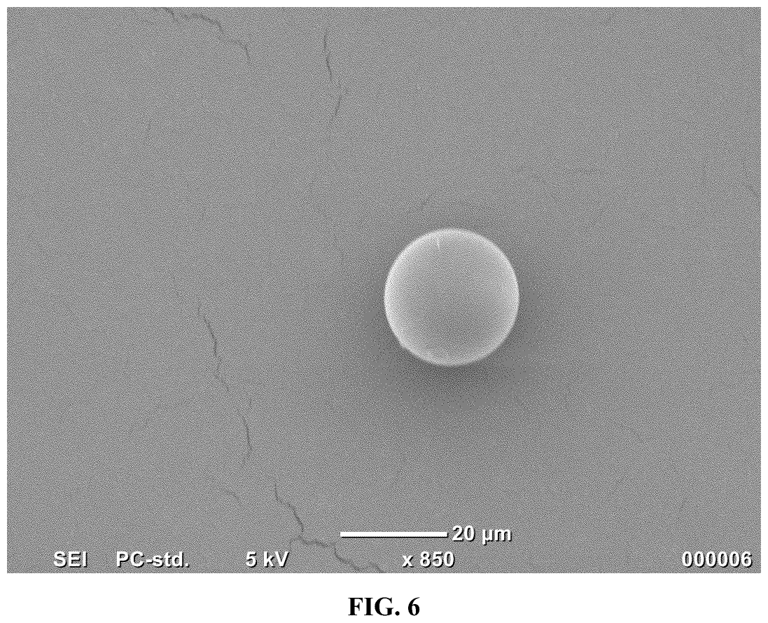

Starting with commercially available hydrogenated poly(limonene) (Clearon P150 (Yasuhara Chemical Co. Ltd, Hiroshima, Japan)), the method of Example 3 was followed to obtain the hydrogenated poly(limonene) particles shown in FIG. 6.

### Example 7: Collection of the Terpene Polymer Particles

The particles obtained in Examples 3-6 were separated from the suspension by either centrifugation or by filtration. Centrifugation was performed at 4500 rpm for 5 minutes. After 5 minutes, the supernatant solution containing polyvinyl alcohol (PVA) was decanted and the particles were suspended in fresh deionized water. Centrifugation was repeated 5 or 6 times to obtain a cake of clean particles free from PVA. After centrifugation, a final filtration using a Millipore filter set was performed followed by vacuum filtration. The resultant wet cake was dried in a vacuum oven at 50° C. for 12 hours to obtain a dry powder of polymer particles.

Comparison of Poly(β-pinene) Particles, Mixed Poly(α-pinene and β-pinene) Particles, Poly(β-pinene), and Commercial Poly(β-pinene)

Morphology, solidity, particle size measurements, and sphericity for the prepared poly(β-pinene) particles of Example 3, for the prepared mixed pinene copolymer particles of Example 4, for the prepared poly(β-pinene) of Example 1, and for a commercially available poly(β-pinene) are provided in TABLE 1. As shown in TABLE 1, the morphology, solidity, volume median diameter, and sphericity differed greatly for the prepared poly(β-pinene) of Example 1, the commercially available poly(β-pinene), the poly(β-pinene) particles of Example 3, and the mixed pinene copolymer particles of Example 4. As discussed herein, in order to achieve the necessary texture and optical effects, polymer particles must be spherical, have a smooth surface that is non-porous, and have a volume median diameter of from about 0.5 μm to about 100 μm. As shown in TABLE 1, the prepared poly(β-pinene) used as a starting material in Example 3 and the commercially available poly(β-pinene) were not spherical, had low solidity, and the individual particles had a volume median diameter of ≥50 μm. After the poly(β-pinene) starting material was subjected to the conditions of Example 3, the resultant particles were found to be spherical, solid, and had a particle volume median diameter from about 5 μm to about 25 μm (i.e., within the desired range). Similarly, after the mixed pinene copolymer starting material was subjected to the conditions of Example 4, the resultant particles were found to be spherical, solid, and had a particle volume median diameter from about 5 μm to about 25 μm (i.e., within the desired range). As discussed herein, the sphericity, solidity, and particle volume median diameter of polymer particles are critical properties for providing successful performance and optimal sensory when used in a beauty and/or cosmetic product.

TABLE 1
Characterization of Poly(β-pinene), Commercial Poly(β-pinene), Poly(β-pinene)
Particles, and Mixed Pinene Copolymer Particles

| | Example 1 | Commercial | Example 3 | Example 4 |
|---|---|---|---|---|
| | Poly(β-pinene) | Poly(β-pinene)*¹ | Particles | Particles |
| Morphology | amorphous lumps | amorphous lumps | spherical, smooth | spherical, smooth |
| Porosity | Porous | Non-porous | Non-porous | Non-porous |
| Particle Volume | Large chunks | Large chunks | 5-25 μm | 5-25 μm |
| Median Diameter Range | of ≥50 μm | of ≥50 μm | | |
| Sphericity | <0.5 | <0.5 | 0.98-1 | 0.98-1 |

*¹= YS Resin PX 1150N (Yashuhara Chemical Co., Ltd., Japan)

Sensory properties for the prepared poly(β-pinene) particles of Example 3, for the prepared mixed pinene copolymer particles of Example 4, for the prepared poly(β-pinene) of Example 1, and for the commercially available poly(β-pinene) are provided in TABLE 3. Sensory data was measured by providing five panelists with a specified amount of the respective sample and instructions to provide an assessment of each sample based on the five listed criteria: spreadability, coarseness, smoothness, glide, smoothness, and whitening (residue). The samples were assessed using the scales and definitions in TABLE 2.

TABLE 2
Definitions and Scales for Attributes.

| Attribute | Definition | Scale |
|---|---|---|
| Spreadability | Ease to spread the product | 1 - Difficult |
| | over the test area | 5 - Easy |
| Coarseness | Rough, grainy skin feel when | 1 - Silky & supple |
| | sliding your finger across the | 5 - Rough |
| | surface with the product | |
| Glide | Ease of moving your finger | 1 - Draggy |
| | with the product smoothly, | 5 - Silky & gliding |
| | continuously, and effortlessly | |
| Smoothness | Overall evaluation of the | 1 - Rough |
| | unevenness and roughness | 5 - Smooth |
| | felt when sliding your finger | |
| | across the surface with the product | |
| Whitening | Degree to which the product | 1 - Translucent |
| | turns white when rubbed in | 5 - Very White |

The average score for the five panelists is provided in TABLE 3.

TABLE 3
Sensory Properties of Poly(β-pinene), Commercial Poly(β-pinene),
Poly(β-pinene) Particles, and Mixed Pinene Copolymer Particles

| | | | | Example 4 |
|---|---|---|---|---|
| | | | Example 3 | Spherical |
| | Example 1 | Commercial | Spherical | particles made |
| | Poly(β- | Poly(β- | particles made | of Commercial |
| | pinene) | pinene)*¹ | of Example 1 | Poly(β-pinene) |
| Spreadability | 1 | 1 | 5 | 5 |
| Coarseness | 5 | 4 | 1 | 2 |
| Glide | 2 | 1 | 4 | 4 |
| Smoothness | 1 | 1 | 5 | 5 |
| Whitening | 4 | 5 | 1 | 2 |

*¹= YS Resin PX 1150N (Yashuhara Chemical Co., Ltd., Japan)

As shown in TABLE 3, the commercially available poly(β-pinene) provided a spreadability of 1/5, a coarseness of 4/5, a glide of 1/5, a smoothness of 1/5, and a whitening of 5/5. The prepared poly(β-pinene) of Example 1 provided a spreadability of 1/5, a coarseness of 5/5, a glide of 2/5, a smoothness of 1/5, and a whitening of 4/5. The prepared poly(β-pinene) particles of Example 3 provided a spreadability of 5/5, a coarseness of 1/5, a glide of 4/5, a smoothness of 5/5, and a whitening of 1/5. And, the prepared mixed pinene copolymer particles of Example 4 provided a spreadability of 5/5, a coarseness of of 2/5, a glide of 4/5, a smoothness of 5/5, and a whitening of 2/5. Thus, the prepared poly(β-pinene) particles and the mixed polypinene particles provide sensory properties that are desirable in personal care compositions and personal care formulations.

### Comparison of Poly(limonene) Particles and Hydrogenated Poly(limonene) Particles

Morphology, solidity, particle size measurements, and sphericity for the prepared poly(limonene) particles of Example 5 and for the prepared hydrogenated poly(limonene) particles of Example 6 are provided in TABLE 4. As discussed herein, in order to achieve the necessary texture and optical effects, polymer particles must be spherical, have a smooth surface that is non-porous, and have a diameter of from about 0.5 μm to about 100 μm. As shown in TABLE 4, after the poly(limonene) starting material was subjected to the conditions of Example 5, the resultant particles were found to be spherical, solid, and had a particle volume median diameter from about 5 μm and about 25 μm (i.e., within the desired range). Similarly, after the hydrogenated poly(limonene) starting material was subjected to the conditions of Example 6, the resultant particles were found to be spherical, solid, and had a particle volume median diameter from about 5 μm and about 100 μm (i.e., within the desired range). As discussed herein, the sphericity, solidity, and particle volume median diameter of polymer particles are critical properties for providing successful performance and optimal sensory when used in a beauty and/or cosmetic product.

TABLE 4
Characterization of Poly(limonene) Particles
and Hydrogenated Poly(limonene) Particles.

| | Example 5 Particles | Example 6 Particles |
|---|---|---|
| Morphology | Spherical, Smooth | Spherical, Smooth |
| Solidity | 100% | 100% |
| Particle Volume Median | 5-100 μm | 5-100 μm |
| Diameter Range | | |
| Sphericity | 1 | 1 |

Comparison of Cosmetic Formulations Using Prepared Poly(β-pinene), Poly(β-pinene) Particles, and Mixed Polypinene Particles

Cosmetic formulations were prepared using the prepared poly(β-pinene) particles of Example 1, the prepared poly(β-pinene) particles of Example 3, and the prepared mixed pinene copolymer particles of Example 4. The ingredients used to prepare the cosmetic formulations are provided in TABLE 5. The cosmetic formulations were prepared by (1) combining the ingredients and mixing until uniform; and (2) adding fragrance as desired and mixing until uniform.

TABLE 5
Cosmetic Formulations Using Prepared Poly(β-pinene), Poly(β-pinene)
Particles, and Mixed Polypinene Particles.

| | | Formulation | |
|---|---|---|---|
| | Formulation | Comprising Mixed | Formulation |
| | Comprising Poly(β- | Pinene Copolymer | Comprising |
| | pinene) Particles | Particles of | Poly(β-pinene) |
| Ingredient | of Example 3 | Example 4 | of Example 1 |
| Example 1 Poly(β- | 20% | — | — |
| pinene) | | | |
| Example 3 Poly(β- | — | 20% | — |
| pinene) Particles | | | |
| Example 4 Mixed | — | — | 20% |
| Pinene Copolymer | | | |
| Particles | | | |
| Crosslinked Polymer | 60% | 60% | 60% |
| Solvent | 20% | 20% | 20% |

Additional sensory properties for formulations comprising the prepared poly(β-pinene) particles of Example 3, for the prepared mixed pinene copolymer particles of Example 4, and for the prepared poly(β-pinene) of Example 1 are provided in TABLE 6. Sensory data was measured by providing five panelists with a specified amount of the respective sample and instructions to provide an assessment of each sample based on the five listed criteria: spreadability, coarseness, smoothness, glide, smoothness, and whitening (residue). The average score for the five panelists is provided in TABLE 6.

TABLE 6
Sensory Properties of Formulations of Poly(β-pinene), Poly(β-pinene)
Particles, and Mixed Pinene Copolymer Particles

| | Example 1 | Example 3 | Example 4 |
|---|---|---|---|
| | Formulation | Formulation | Formulation |
| Spreadability | 1 | 5 | 5 |
| Coarseness | 5 | 1 | 2 |
| Glide | 2 | 4 | 4 |
| Smoothness | 1 | 5 | 5 |
| Whitening | 4 | 1 | 2 |

As shown in TABLE 6, a formulation comprising the prepared poly(β-pinene) of Example 1 provided a spreadability of 2/5, a coarseness of 5/5, a glide of 2/5, a smoothness of 1/5, and a whitening of 4/5. A formulation comprising the prepared poly(β-pinene) particles of Example 3 provided a spreadability of 5/5, a coarseness of 1/5, a glide of 5/5, a smoothness of 5/5, and a whitening of 1/5. And, a formulation comprising the prepared mixed pinene copolymer particles of Example 4 provided a spreadability of 5/5, a coarseness of 1/5, a glide of 5/5, a smoothness of 5/5, and a whitening of 1/5. Thus, a formulation comprising poly(β-pinene) shows decreased spreadability, increased coarseness, decreased glide, decreased smoothness, and an increased whitening effect when applied to the skin compared to formulations comprising the prepared poly(β-pinene) particles and the mixed pinene copolymer particles.

The following examples are included to demonstrate personal care formulations containing particles of the invention. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific examples which are disclosed and still obtain a like or similar result without departing from the spirit and scope of the disclosure.

### Formulation Example 1: Powder Foundation

Powder foundations were prepared using the ingredients listed in TABLE 7 including the poly(β-pinene) particles of Example 3. The ingredients of Phase A and Phase B were mixed in an IKA Lab Mixer (IKA Works, Inc., Wilmington, NC) then the premixed ingredients in Phase C were added. The mixture was mixed until uniform using the IKA Lab Mixer. Then the mixture was compression molded into a metal plate.

TABLE 7
Powder Foundation Formulation

| | | Amount |
|---|---|---|
| | Ingredient | (Wt. %) |
| Phase | Poly(β-pinene) particles of Example 3 | 10 |
| A | Polymethylhydrosiloxane treated Talc | 22 |
| | Polymethylhydrosiloxane treated Mica | 33 |
| | Polymethylhydrosiloxane treated Titanium Dioxide | 11.5 |
| | Polymethylhydrosiloxane treated Zinc Oxide | 3 |
| | Boron Nitride Powder | 4 |
| | Microsphere M-305 (Poly(methyl methacrylate) | 1 |
| | particles) | |
| | (Matsumoto Yushi-Seiyaku Co. Ltd, Yao, Japan) | |
| | Magnesium Stearate | 2 |
| Phase | Polymethylhydrosiloxane treated Iron Oxides (CI* | 2.2 |
| B | 77492) | |
| | Polymethylhydrosiloxane treated Iron Oxides (CI* | 0.54 |
| | 77491) | |
| | Polymethylhydrosiloxane treated Iron Oxides (CI* | 0.36 |
| | 77499) | |
| Phase | Polydimethylsiloxane trimethylsiloxysilicate | 2 |
| C | (SS4267, Momentive Performance Materials, | |
| | Niskayuna, New York) | |
| | Pentylene Glycol | 2 |
| | SILSOFT B3820 (isododecance and dimethicone blend) | 1 |
| | (Momentive Performance Materials, Niskayuna, | |
| | New York) | |
| | Caprylic/Capric Triglyceride | 1.6 |
| | Sorbitan Sesquiisostearate | 0.5 |
| | Diisostearyl Malate | 0.5 |
| | Ethylhexyl Methoxycinnamate | 2 |
| | Tocopheryl Acetate | 0.5 |
| | Phenoxylethanol | 0.3 |

*CI = Color Index Constitution Number

### Formulation Example 2: Powder Formulation

Powder formulations were prepared using the ingredients listed in TABLE 8 including the poly(β-pinene) particles of Example 3. The ingredients were mixed in a 100 gm SPEEDMIXER container (FLACKTEK, Louisville, CO) and the resultant powder was mixed for 5 minutes to obtain a uniformly mixed loose powder formulation.

TABLE 8
Powder Formulation

| | | Amount |
|---|---|---|
| | Ingredient | (Wt. %) |
| 1 | Talc and Triethoxycaprylylsilane | 23.4 |
| 2 | Mica and Triethoxycaprylylsilane | 35.1 |
| 3 | Titanium Dioxide, Alumina, and Methicone | 12.2 |
| 4 | Zinc Oxide and Triethoxycaprylylsilane | 3.2 |
| 5 | Boron Nitride | 4.3 |
| 6 | CI* 77492 and Isopropyl Titanium Triisostearate | 2.3 |
| 7 | CI* 77491 and Isopropyl Titanium Triisostearate | 0.6 |
| 8 | CI 77499 and Isopropyl Titanium Triisostearate | 0.4 |
| 9 | Dimethicone and Trimethylsiloxysilicate | 2.1 |
| 10 | Caprylic/Capric Triglyceride | 2.8 |
| 11 | Sorbitan Sesquiisostearate | 0.5 |
| 12 | Diisostearyl Malate | 0.5 |
| 13 | Phenoxyethanol | 0.3 |
| 14 | Ethylhexyl Methoxycinnamate | 2.1 |
| 15 | Poly(β-pinene) particles of Example 3 | 10.0 |

*CI = Color Index Constitution Number

### Formulation Example 3: Emulsified Foundation Formulation

An emulsified foundation formulation was prepared using the ingredients listed in TABLE 9 including the poly(β-pinene) particles of Example 3. The preparation method was as follows: first, ingredients (5) to (8) were mixed with an IKA Lab Mixer, then ingredients (1) and (3) to (4) were added thereto, and the mixture was thoroughly mixed, and then pulverized and classified. Further, ingredients (2) and (9) to (18) were added, treated with a homomixer, deaerated, and filled into a container.

TABLE 9
Emulsified Foundation Formulation

| | Ingredient | Amount (Wt. %) |
|---|---|---|
| 1 | Poly(β-pinene) particles of Example 3 | 10.0 |
| 2 | Dimethyl silicone (viscosity 10 mPa · s) | 7.0 |
| 3 | Titanium oxide | 5.0 |
| 4 | Anhydrous silicic acid | 3.0 |
| 5 | Talc | 8.0 |
| 6 | Bengala | 1.0 |
| 7 | Black iron oxide | 0.5 |
| 8 | Yellow iron oxide | 1.0 |
| 9 | Octamethylcyclotetrasiloxane | 10.0 |
| 10 | Rosin pentaerislit ester | 2.0 |
| 11 | Neopentyl glycol diisooctanoate | 4.0 |
| 12 | Squalene | 2.5 |
| 13 | Glycerin triisooctanoate | 2.0 |
| 14 | Purified water | 35.0 |
| 15 | 1,3-butylene glycol | 4.0 |
| 16 | Ethanol | 8.0 |
| 17 | Preservatives | 0.1 |
| 18 | Fragrance | Balance |

### Formulation Example 4: Powder Foundation Formulation

A dual-purpose powder foundation formulation was prepared using the ingredients listed in TABLE 10 including the mixed pinene copolymer particles of Example 4. The preparation method was as follows: first, ingredients (1) and (3) to (10) were mixed and pulverized, transferred to an IKA mixer, and then ingredients (2) and (11) to (16) were added and mixed uniformly. Then the mixture was compression molded into a metal plate.

TABLE 10
Powder Foundation Formulation

| | Ingredient | Amount (Wt. %) |
|---|---|---|
| 1 | Mixed pinene copolymer particles of Example 4 | 15.0 |
| 2 | Dimethyl silicone (viscosity 10 mPa · s) | 5.0 |
| 3 | Mica | 5.0 |
| 4 | Talc | 5.0 |
| 5 | Titanium oxide | 15.0 |
| 6 | Mica Titanium | 3.5 |
| 7 | Iron oxide (red, yellow, black) | 7.0 |
| 8 | Zinc oxide | 4.0 |
| 9 | Aluminum oxide | 10.0 |
| 10 | Barium sulfate | 5.0 |
| 11 | Lanolin | 5.0 |
| 12 | Vaseline | 1.5 |
| 13 | Liquid paraffin | 1.0 |
| 14 | Isopropyl millistate | 1.5 |
| 15 | Preservatives | Balance |
| 16 | Fragrance | Balance |

### Formulation Example 5: Powder Eyeshadow Formulation

A powder eyeshadow formulation was prepared using the ingredients listed in TABLE 11 including the poly(limonene) particles of Example 5. The preparation method was as follows: first, ingredients (1) and (3) to (10) were mixed and crushed, transferred to a Henschel mixer, then ingredients (2) and (11) and (12) were added and mixed uniformly, then the mixture was compression molded into a metal plate.

TABLE 11
Powder Eyeshadow Formulation

| | Ingredient | Amount (Wt. %) |
|---|---|---|
| 1 | Poly(limonene) particles of Example 5 | 20.0 |
| 2 | Dimethyl silicone (viscosity 10 mPa · s) | 5.0 |
| 3 | Mica | Balance |
| 4 | Talc | 15.0 |
| 5 | Mica Titanium | 8.0 |
| 6 | Zinc stearate | 4.0 |
| 7 | Zinc laurate | 4.0 |
| 8 | Yellow iron oxide | 0.7 |
| 9 | Black iron oxide | 0.7 |
| 10 | Red iron oxide | 0.7 |
| 11 | Liquid paraffin | 8.0 |
| 12 | Preservatives and fragrances | Balance |

### Formulation Example 6: Two-Layer Sunscreen Formulation

A two-layer sunscreen formulation was prepared using the ingredients listed in TABLE 12 including the poly(β-pinene) particles of Example 3. The preparation method was as follows: ingredients (1) to (7) were first dispersed and mixed with a homogenizer, and then the aqueous phase ingredients (8) to (13) were added and stirred to emulsify.

TABLE 12
Two-Layer Sunscreen Formulation

| | Ingredient | Amount (Wt. %) |
|---|---|---|
| 1 | Poly(β-pinene) particles of Example 3 | 10.0 |
| 2 | Dimethyl silicone (viscosity 10 mPa · s) | 5.0 |
| 3 | Decamethylcyclopentasiloxane | 20.0 |
| 4 | Polyether-modified silicone | 1.0 |
| 5 | Squalene | 8.0 |
| 6 | Hydrophobicized titanium oxide | 5.0 |
| 7 | Octyl methoxycinnamic acid | 7.0 |
| 8 | Glycerin | 2.0 |
| 9 | Sodium Chloride | 0.40 |
| 10 | Polysorbate 20 | 0.90 |
| 11 | Ethanol | 5.0 |
| 12 | Fragrance | Balance |
| 13 | Purified water | Balance |

### Formulation Example 7: Powder Foundation Formulation

A sunscreen cream was prepared using the ingredients listed in TABLE 13 including the poly(β-pinene) particles of Example 3. The preparation method was as follows: ingredients (1) to (8) were first dispersed and mixed with a homogenizer, and then the aqueous phase ingredients (9) to (10) were added and stirred to emulsify.

TABLE 13
Sunscreen Cream Formulation

| | Ingredient | Amount (Wt. %) |
|---|---|---|
| 1 | Poly(β-pinene) particles of Example 3 | 10.0 |
| 2 | Dimethyl silicone (viscosity 10 mPa · s) | 7.0 |
| 3 | Hydrophobic titanium dioxide | 10.0 |
| 4 | Hydrophobic zinc oxide | 10.0 |
| 5 | Squalene | 15.0 |
| 6 | Glycerin diisostearate | 3.0 |
| 7 | Preservatives | 0.1 |
| 8 | Fragrance | 0.1 |
| 9 | Purified water | Balance |
| 10 | 1,3-butylene glycol | 5.0 |

### Formulation Example 8: Solid White Powder Formulation

A solid white powder formulation was prepared using the ingredients listed in TABLE 14 including the hydrogenated poly(limonene) particles of Example 6. The preparation method was as follows: first, ingredients (1) and (3) to (6) were mixed and crushed, transferred to a Henschel mixer, then ingredients (2) and (7) to (10) were added, mixed uniformly, and compression molded into a metal plate.

TABLE 14
Solid White Powder Formulation

| | Ingredient | Amount (Wt. %) |
|---|---|---|
| 1 | Hydrogenated poly(limonene) particles of | 20.0 |
| | Example 6 | |
| 2 | Dimethyl silicone (viscosity 10 mPa · s) | 7.0 |
| 3 | Mica Balance | Balance |
| 4 | Talc | 15.0 |
| 5 | Titanium oxide | 1.0 |
| 6 | Yellow iron oxide | 1.0 |
| 7 | Liquid paraffin | 10.0 |
| 8 | Beeswax | 3.0 |
| 9 | Preservatives | Balance |
| 10 | Fragrance | Balance |

### Formulation Example 9: Blusher Formulation

A blusher formulation was prepared using the ingredients listed in TABLE 15 including the poly(limonene) particles of Example 5. The preparation method was as follows: first, ingredients (1) to (6) were mixed and crushed, transferred to an IKA mixer, then the ingredients (7) to (10) were added, mixed uniformly, and compression molded into a metal plate.

TABLE 15
Blusher Formulation

| | Ingredient | Amount (Wt. %) |
|---|---|---|
| 1 | Poly(limonene) particles of Example 5 | 5.0 |
| 2 | Mica | 10.0 |
| 3 | Titanium oxide | 10.0 |
| 4 | Red iron oxide | 1.5 |
| 5 | Black iron oxide | 1.5 |
| 6 | Yellow iron oxide | 1.5 |
| 7 | Squalene | 7.0 |
| 8 | Dimethyl silicone (viscosity 5 mPa · s) | 7.0 |
| 9 | Preservatives | 0.1 |
| 10 | Fragrance | Balance |

### Formulation Example 10: Lipstick Formulation

A lipstick formulation was prepared using the ingredients listed in TABLE 16 including the mixed pinene copolymer particles of Example 4. The preparation method was as follows: ingredients (1)-(11) were heated and melted, then (12) and (13) were added and mixed, the mixture was degassed and poured into a container, rapidly cooled, and hardened.

TABLE 16
Lipstick Formulation

| | Ingredient | Amount (Wt. %) |
|---|---|---|
| 1 | Mixed pinene copolymer particles of Example 4 | 6.0 |
| 2 | Dimethyl silicone (viscosity 10 mPa · s) | 10.0 |
| 3 | Paraffin wax | 11.0 |
| 4 | Lanolin wax | 12.0 |
| 5 | Candelilla wax | 5.0 |
| 6 | Beeswax | 5.0 |
| 7 | Castor oil | Balance |
| 8 | Glycerin trioctanoate | 2.0 |
| 9 | Titanium oxide | 1.0 |
| 10 | Red No. 201 | 3.0 |
| 11 | Blue No. 1 aluminum lake | 0.5 |
| 12 | Preservatives | Balance |
| 13 | Fragrance | Balance |

### Formulation Example 11: BB Cream Formulation

A BB cream formulation was prepared using the ingredients listed in TABLE 17 including the poly(β-pinene) particles of Example 3. The preparation method was as follows: ingredients (1) to (9) were mixed until uniform, ingredients (10) to (15) were separately mixed well, then added to ingredients (1) to (9) and mixed well, then the aqueous phase ingredients (16) to (21) were added and mixed well, and finally added ingredient (22) and homogenized to uniformity.

TABLE 17
BB Cream Formulation

| | Ingredients | Amount (Wt. %) |
|---|---|---|
| 1 | Caprylyl Methicone and C30-45 Alkyl Cetearyl | 4 |
| | Dimethicone Crosspolymer | |
| 2 | Dimethicone | 15 |
| 3 | Poly(β-pinene) particles of Example 3 | 5 |
| 4 | Trifluoropropyldimethylsiloxy/Trimethylsiloxy | 2 |
| | Silsesquioxane and Dimethicone | |
| 5 | (Caprylic/Capric) Triglyceride and PEG/ | 7 |
| | PPG-20/15 Dimethicone | |
| 6 | Bisphenylpropyl Dimethicone | 2 |
| 7 | Boron Nitride | 3 |
| 8 | Phenoxyethanol and Ethylhexylglycerin | 1 |
| 9 | Fragrance | Balance |
| 10 | Caprylyl Methicone | 5 |
| 11 | Titanium Dioxide and Triethoxycaprylylsilane | 2 |
| 12 | Iron Oxide and Triethoxycaprylylsilane | 0.5 |
| 13 | Iron Oxide and Triethoxycaprylylsilane | 0.2 |
| 14 | Iron Oxide and Triethoxycaprylylsilane | 0.05 |
| 15 | Titanium Dioxide, Alumina, and Methicone | 7 |
| 16 | Water | Balance |
| 17 | Trisodium EDTA | 0.2 |
| 18 | Butylene Glycol | 3 |
| 19 | Glycerin | 5 |
| 20 | Sodium Chloride | 1 |
| 21 | Hydroxyphenyl Propamidobenzoic Acid | 1 |
| 22 | Disteardimonium Hectorite | 0.7 |

### Formulation Example 12: CC Cream Formulation

A CC cream formulation was prepared using the ingredients listed in TABLE 18 including the poly(β-pinene) particles of Example 3. The preparation method was as follows: ingredients (5) to (14) were mixed until uniform, ingredients (15) to (17) were separately mixed well, then added to ingredients (5) to (14) and mixed well, then the aqueous phase ingredients (1) to (4) were added and mixed well, finally added (18) to (20), and mixed to uniformity.

TABLE 18
CC Cream Formulation

| | | Amount |
|---|---|---|
| | Ingredients | (Wt. %) |
| 1 | Water | Balance |
| 2 | Glycerin | 5 |
| 3 | Sodium Chloride | 1 |
| 4 | Propylene Glycol | 8 |
| 5 | PEG-9 Dimethicone | 1 |
| 6 | Caprylic/Capric Triglyceride and PEG/PPG-20/15 | 3 |
| | Dimethicone | |
| 7 | Caprylyl Methicone | 12 |
| 8 | Diphenyl Dimethicone | 5 |
| 9 | Boron Nitride | 1 |
| 10 | Poly(β-pinene) particles of Example 3 | 5 |
| 11 | Polymethylsilsesquioxane | 1 |
| 12 | Octyl MethoxyCinnamate | 4 |
| 13 | Titanium Dioxide and Methylhydrogenpolysiloxane | 5 |
| 14 | Disteardimonium Hectorite | 0.5 |
| 15 | Yellow Iron Oxide and Methylhydrogenpolysiloxane | 0.25 |
| 16 | Red Iron Oxide and Methylhydrogenpolysiloxane | 0.15 |
| 17 | Black Iron Oxide and Methylhydrogenpolysiloxane | 0.08 |
| 18 | Dimethicone/Vinyl Dimethicone Crosspolymer, | 5 |
| | Dimethicone, Isohexadecane, and Cetearyl Methicone | |
| 19 | Fragrance | Balance |
| 20 | Phenoxyethanol, Chlorphenesin, and Glycerin | Balance |

### Formulation Example 13: Mascara Formulation

A mascara formulation was prepared using the ingredients listed in TABLE 19 including the poly(β-pinene) particles of Example 3. The preparation method was as follows: ingredients (3) and (4) were mixed in water then heated to uniformity, added ingredients (2) and (5) to (9) and mixed well, heated ingredients (10) to (15) separately, then added to the mixture and mixed well, then cooled to 45° C., added ingredients (16) and (17) one by one, and mixed to uniformity.

TABLE 19
Mascara Formulation

| | Ingredients | Amount (Wt. %) |
|---|---|---|
| 1 | Water | Balance |
| 2 | Poly(β-pinene) particles of Example 3 | 5 |
| 3 | Polyvinylpyrrolidone | 2 |
| 4 | Hydroxyethylcellulose | 1 |
| 5 | Triethanolamine | 1 |
| 6 | Methylparaben | 0.3 |
| 7 | Disodium EDTA | 0.1 |
| 8 | Black Iron Oxide | 10 |
| 9 | Dimethicone PEG-8 Polyacrylate | 7.2 |
| 10 | Stearic Acid | 4.5 |
| 11 | Glyceryl Monostearate | 2 |
| 12 | White Bleached Beeswax | 7 |
| 13 | Carnauba Wax | 4.5 |
| 14 | Hydroxylated Lanolin | 1 |
| 15 | Propylparaben | Balance |
| 16 | Acrylates Copolymer | 20 |
| 17 | DMDM Hydantoin | Balance |

### Formulation Example 14: Concealer Formulation

A concealer formulation was prepared using the ingredients listed in TABLE 20 including the hydrogenated poly(limonene) particles of Example 6. The preparation method was as follows: ingredients (1) and (7) were mixed until uniform and heated to 90° C., added ingredients (8) to (10) and mixed until uniform, and poured the mixture into suitable container.

TABLE 20
Concealer Formulation

| | | Amount |
|---|---|---|
| | Ingredients | (Wt. %) |
| 1 | Boron Nitride | 10 |
| 2 | Hydrogenated poly(limonene) particles of Example 6 | 5 |
| 3 | Dimethicone | Balance |
| 4 | Titanium Dioxide and Triethoxycaprylylsilane | 4 |
| 5 | Iron Oxide and Triethoxycaprylylsilane | 0.9 |
| 6 | Iron Oxide and Triethoxycaprylylsilane | 0.3 |
| 7 | Iron Oxide and Triethoxycaprylylsilane | 0.1 |
| 8 | Ozokerite Wax | 5 |
| 9 | Polyethylene | 5 |
| 10 | Synthetic Wax and Microcrystalline Wax | 2 |

### Formulation Example 15: Oil-in-Water Cream Formulation

A oil-in-water (O/W) cream formulation was prepared using the ingredients listed in TABLE 21 including the poly(β-pinene) particles of Example 3. The preparation method was as follows: ingredients (1) to (3) were mixed and heated to 80° C., ingredients (4) to (6) were mixed separately and added to ingredients (1) to (3), mixed until uniform, cooled to room temperature, and added ingredients (7) and (8) and mixed until uniform.

TABLE 21
O/W Cream Formulation

| | | Amount |
|---|---|---|
| | Ingredients | (Wt. %) |
| 1 | Water | Balance |
| 2 | Poly(β-pinene) particles of Example 3 | 9.5 |
| 3 | Titanium Dioxide, Silica, and Aluminum Hydroxide | 0.5 |
| 4 | Polysilicone-34, Isononyl Isononanoate, and Water | 3 |
| 5 | Dimethicone | 8 |
| 6 | Butyrospermum Parkii (Shea) Butter | 2 |
| 7 | Phenoxyethanol | Balance |
| 8 | Sodium Hyaluronate | 0.1 |

### Formulation Example 16: W/O Cream Formulation

A water-in-oil (W/O) cream formulation was prepared using the ingredients listed in TABLE 22 including the mixed pinene copolymer particles of Example 4. The preparation method was as follows: ingredients (1) to (6) were mixed and heated to 70° C., ingredients (7) to (9) were mixed separately and added to ingredients (1) to (6), mixed to emulsify, and cooled to room temperature.

TABLE 22
W/O Cream Formulation

| | | Amount |
|---|---|---|
| | Ingredients | (Wt. %) |
| 1 | Dimethicone | 1 |
| 2 | Capylyl Methicone | 10 |
| 3 | Mixed pinene copolymer particles of Example 4 | 9 |
| 4 | Hydrogen Dimethicone, Titanium Dioxide, and | 1 |
| | Aluminum Hydroxide | |
| 5 | (Caprylyl/Capryl) Triglyceride and PEG/PPG-20/15 | 3 |
| | Dimethicone | |
| 6 | Phenoxy ethanol | Balance |
| 7 | Water | Balance |
| 8 | Glycerin | 8 |
| 9 | Sodium Chloride | 1 |

### Formulation Example 17: Eye Cream Formulation

An eye cream formulation was prepared using the ingredients listed in TABLE 23 including the poly(β-pinene) particles of Example 3. The preparation method was as follows: ingredients (1) to (4) were mixed and heated to 70° C., ingredients (5) to (12) were mixed separately and heated to 70° C., then added to ingredients (1) to (4), mixed to emulsify, then cooled to 50° C., added ingredient (13) and mixed until uniform.

TABLE 23
Eye Cream Formulation

| | | Amount |
|---|---|---|
| | Ingredients | (Wt. %) |
| 1 | Water | Balance |
| 2 | Poly(β-pinene) particles of Example 3 | 5 |
| 3 | Glycerin | 3 |
| 4 | Disodium EDTA | 0.05 |
| 5 | Glyceryl stearate citrate, Polyglyceryl-3 stearate, | 4 |
| | and Hydrogenated lecithin | |
| 6 | Sodium acrylates copolymer and Lecithin | 1 |
| 7 | Cetyl Alcohol | 1.5 |
| 8 | Butyrospermum Parkii (Shea Butter) | 4 |
| 9 | Decyl Isostearate and Isostearyl Isostearate | 3 |
| 10 | Argania Spinosa Kernel Oil | 2 |
| 11 | Dimethicone | 3 |
| 12 | Caprylyl Methicone | 3 |
| 13 | Dimethicone/Vinyl Dimethicone Crosspolymer, | 3 |
| | Dimethicone, Isohexadecane, and Cetearyl Methicone | |

### Formulation Example 18: Skin Serum Formulation

A skin serum formulation was prepared using the ingredients listed in TABLE 24 including the poly(β-pinene) particles of Example 3. The preparation method was as follows: ingredients (1) to (4) were mixed until uniform, ingredients (5) to (7) were mixed separately and added to ingredients (1) to (4), mixed until uniform, then added ingredients from (8) to (11) one by one, and mixed until uniform.

TABLE 24
Skin Serum Formulation

| | | Amount |
|---|---|---|
| | Ingredients | (Wt. %) |
| 1 | Water | Balance |
| 2 | Poly(β-pinene) of Example 3 | 1 |
| 3 | Alpha-Glucan Oligosaccharide | 0.5 |
| 4 | Hydrolyzed Sodium Hyaluronate | 0.01 |
| 5 | BG | 1 |
| 6 | Pentylene Glycol | 3 |
| 7 | Dipropylene Glycol and Polysilicone-29 | 1 |
| 8 | Malus Domestica Fruit Cell Culture Extract | 0.1 |
| 9 | Ascorbyl Tetraisopalmitate | 0.1 |
| 10 | Phenoxyethanol | Balance |
| 11 | Fragrance | Balance |

### Formulation Example 19: Shampoo Formulation

A shampoo formulation was prepared using the ingredients listed in TABLE 25 including the poly(β-pinene) particles of Example 3. The preparation method was as follows: added ingredients (3) to water, mixed uniform and heated to 80° C., after the mixture was clear, added ingredients (2) and (5) to (10) to the mixture one by one and confirmed the mixture was clear, cooled to room temperature, and added ingredients (11) to (15) one by one and mixed until uniform.

TABLE 25
Shampoo Formulation

| | Ingredients | Amount (Wt. %) |
|---|---|---|
| 1 | Water | Balance |
| 2 | Dipropylene Glycol | 3 |
| 3 | Polyquaternium 10 | 0.4 |
| 5 | Poly(β-pinene) particles of Example 3 | 5 |
| 6 | Disodium EDTA | 0.05 |
| 7 | Sodium Benzoate | 0.5 |
| 8 | Sodium Laureth Sulfate | 17 |
| 9 | Cocamidopropyl Betaine | 10 |
| 10 | Cocamide MEA | 2 |
| 11 | Phenoxyethanol | Balance |
| 12 | Water, Glycol Distearate, Glycerin, | 5 |
| | Laureth-4, and Cocamidopropyl Betain | |
| 13 | Citric acid | 0.1 |
| 14 | Dimethiconol, Water, Sodium Lauryl | 2 |
| | Sulfate, and Sodium Laureth Sulfate | |
| 15 | Fragrance | Balance |

### Formulation Example 20: Skin Primer Formulation

A skin primer formulation was prepared using the ingredients listed in TABLE 26 including the poly(limonene) particles of Example 5. The preparation method was as follows: ingredients (1) to (7) were heated and mixed, ingredients (8) to (13) were separately mixed well, then added to ingredients (1) to (7) and mixed well, finally the aqueous phase ingredients (14) to (18) were added and stirred to emulsify.

TABLE 26
Skin Primer Formulation

| | | Amount |
|---|---|---|
| | Ingredients | (Wt. %) |
| 1 | Dimethicone | 10.5 |
| 2 | Ethylhexyl Methoxycinnamate | 5 |
| 3 | Poly(limonene) particles of Example 5 | 5 |
| 4 | Cylcopentasiloxane and Polymethylsilsesquioxane | 4 |
| 5 | Dimethicone and Cetearyl Dimethicone Crosspolymer | 4 |
| 6 | Sorbitan Sesquiisostearate | 0.5 |
| 7 | (Caprylic/Capric) Triglyceride and PEG/PPG-20/15 | 4 |
| | Dimethicone | |
| 8 | Dimethicone | 3 |
| 9 | Hydrogen Dimethicone and Zinc Oxide | 1.5 |
| 10 | Hydrogen Dimethicone, Titanium Dioxide, and | 1.5 |
| | Aluminum Hydroxide | |
| 11 | Methicone and Iron Oxide (CI* 77492) | 0.05 |
| 12 | Methicone and Iron Oxide (CI* 77491) | 0.05 |
| 13 | Methicone and Iron Oxide (CI* 77499) | 0.015 |
| 14 | Water | QS |
| 15 | Glycerin | 5 |
| 16 | Polysorbate 20 | 0.5 |
| 17 | Sodium Chloride | 1 |
| 18 | Phenoxy ethanol | 0.01 |

*CI = Color Index Constitution Number

### OTHER ASPECTS

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific aspects thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and can be applied to the essential features hereinbefore set forth, and follows in the scope of the claims.

1. A composition comprising polymer particles, wherein the polymer particles comprise at least one terpene polymer and wherein the polymer particles have a sphericity of at least 0.95 and a Dv50 particle size from about 0.5 μm to about 100 μm.

2. The composition of claim 1, wherein the at least one terpene polymer is obtained by polymerizing at least one terpene monomer selected from the group consisting of a monoterpene, a sesquiterpene, a diterpene, a sesterterpene, a triterpene, a sesquaterpene, a tetraterpene, and combinations thereof.

3. The composition of claim 2, wherein the at least one terpene monomer is a monoterpene selected from the group consisting of myrcene, ocimene, alloocimene, L-limonene, D-limonene, α-pinene, β-pinene, and combinations thereof.

4. The composition of claim 1, wherein the at least one terpene polymer is a homopolymer obtained by polymerizing α-pinene, β-pinene, L-limonene, or D-limonene.

5. The composition of claim 1, wherein the at least one terpene polymer is a copolymer obtained by polymerizing at least two terpene monomers.

6. The composition of claim 5, wherein the at least one terpene polymer is a copolymer obtained by polymerizing α-pinene, β-pinene, L-limonene, D-limonene, or combinations thereof.

7. The composition of claim 5, wherein the at least one terpene polymer is a copolymer obtained by polymerizing at least one terpene monomer and at least one monomer that is not a terpene.

8. The composition of claim 1, wherein the at least one terpene polymer is hydrogenated from about 0.1% to 100%.

9. The composition of claim 1, wherein the at least one terpene polymer has a weight average molecular weight of greater than or equal to about 200 g/mol.

10. (canceled)

11. The composition of claim 1, wherein the at least one terpene polymer has a softening point of greater than or equal to about 50° C.

12. (canceled)

13. The composition of claim 1, wherein the polymer particles have a Dv50 particle size from about 1 μm to about 50 μm.

14. The composition of claim 1, wherein the polymer particles have a sphericity of at least 0.98.

15. The composition of claim 1, wherein the polymer particles are hollow.

16. The composition of claim 1, wherein the polymer particles are solid.

17. The composition of claim 1, wherein the polymer particles are porous.

18. The composition of claim 1, wherein the polymer particles are non-porous.

19. The composition of claim 1, wherein the polymer particles are smooth.

20. The composition of claim 1, wherein the polymer particles are insoluble in non-polar oils.

21. The composition of claim 1, formulated for application to a keratinous substrate, to a fabric, or to leather.

22. A personal care composition comprising the composition of polymer particles of claim 1.

23. The personal care composition of claim 22, wherein the personal care composition is anhydrous.

24. The personal care composition of claim 22, wherein the personal care composition is an oil-in-water or a water-in-oil emulsion.

25. The personal care composition of claim 22, wherein the personal care composition is a powder composition.

26. The personal care composition of claim 22, further comprising at least one additional component selected from the group consisting of pigments, particulates, dyes, actives, fragrances, resins, waxes, cross-linked elastomers, emulsifiers, and combinations thereof.

27. A personal care formulation comprising the composition of polymer particles of claim 1, wherein the personal care formulation is selected from the group consisting of a deodorant, an antiperspirant, a skin cream, a facial cream, a hair shampoo, a hair conditioner, a mousse, a hair styling gel, a hair spray, a protective cream, a lipstick, a facial foundation, blushes, makeup, a mascara, a skin care lotion, a moisturizer, a facial treatment, a personal cleanser, a facial cleanser, a bath oil, a perfume, a shaving cream, a pre-shave lotion, an after-shave lotion, a cologne, a sachet, and a sunscreen.

28. (canceled)
